(19) United States
(12) Patent Application Publication
(10) Pub. No.: US 2006/0206010 A1
(43) Pub. Date: Sep. 14, 2006

(54) Diagnostic support system and mobile terminal

(75) Inventors: Kazuhiro Iida, Tokyo (JP); Toru Sano, Tokyo (JP); Wataru Hattori, Tokyo (JP)

Correspondence Address: FOLEY AND LARDNER LLP;SUITE 500, 3000 K STREET NW, WASHINGTON, DC, 20007, US

(73) Assignee: NEC CORPORATION

(21) Appl. No.: 10/554,532

(22) PCT Filed: May 7, 2004
(86) PCT No.: PCT/JP04/06034

§ 371 Date: Oct. 26, 2005

(30) Foreign Application Priority Data
May 9, 2003 (JP) 2003-132382

(51) Int. Cl. A61B 5/00

(52) U.S. Cl. 600/300; 128/920

(57) ABSTRACT

A diagnostic support system includes a mobile terminal (14) and an analytical center (20), which are connected to each other through a network, and judges a morbidity possibility that a user holding the mobile terminal (14) is suffering from a disease. The analytical center (20) includes a data obtaining unit (26) which obtains symptom data representing the symptom of the user from the mobile terminal (14) in correspondence with a position at which and a date on which the symptom data was made, a morbidity possibility calculation unit (36) which judges the morbidity possibility that the user is suffering from the disease, based on the symptom data and a reference parameter representing the feature generated in sufferers of the disease, and an estimation processing unit (34) which estimates the existing states of sufferers of the disease for each area and in each period, based on morbidity possibilities of a plurality of users, and corresponding positions and dates.

### TECHNICAL FIELD

The present invention relates to a diagnostic support system which judges a morbidity possibility of a disease, the developing degree of which depends on positions and times.

### BACKGROUND ART

Recently, a number of sufferers of an allergic disease such as pollinosis (hay fever) have been increasing, wherein the sufferers of the allergic disease are annoyed with snivel-running, watery eye, sneezing, or itching when pollens of trees, grasses, weeds, and the like enter into noses and eyes of the sufferers. When the pollens enter into a body, antigens (allergens) causing allergic manifestation solve out from the pollens. White blood cells produce antibodies (IgE antibodies) by immune reaction to the antigens. When the same antigens enter into the body again, the antibodies make mast cells release histamines and the like as allergic causative substances. When the allergic causative substances such as histamines stimulate nerves and cells, the above-described symptoms of pollinosis are caused.

The above symptoms of pollinosis are very similar to those of an incipient cold, and seasons in which cedar pollens, which have been well known as a cause of pollinosis, are scattered overlap seasons in which the cold is widespread from the end of January to about April. Moreover, even a person who has never suffered from pollinosis up to a year earlier may be annoyed with pollinosis because anybody can develop pollinosis when antibodies are accumulated in the body in a saturated state.

Furthermore, the developing degree of the allergic disease such as pollinosis is changed not only by the body constitution of a patient, but also by the amount of the causative substances (pollens). Accordingly, sufferers of pollinosis repeat development and remission of the allergy many times in the season in which the pollens are scattering, and the sufferers are required to determine whether the symptoms are caused by the allergy or by other diseases such as the cold, whenever the sufferers develop the allergy. However, it is difficult even for the sufferers of pollinosis to make the above determination.

For example, a method in which an inflammatory response is observed by putting a seal, to which antigens are applied, on the skin of a test subject, by giving an injection of antigens to a test subject, or the like; inspection for presence of the IgE antibody included in the body fluids of a patient; and inspection for presence of eosinophils included in the body fluids of a patient have been known as a diagnostic method by which it is determined whether a person is suffering from pollinosis or not. A serum is often used as a body fluid for the inspection for presence of the IgE antibody included in the body fluids of a patient. A method in which eosinophils are observed with a microscope after snivel is applied on a slide glass for staining, and the number of the eosinophils is counted has been known as a method for inspecting the presence of eosinophils. The cold can be distinguished from pollinosis, based on the above eosinophil inspection because mainly the number of neutrophils is increased in the case of a cold.

Moreover, antigens causing the allergy can be retrieved even by measuring the amount of histamine which is liberated in the body fluids of a patient by stimulating the antigens. Patent document 1 has disclosed that a sensor, which measures the amount of histamine, can be realized by using enzymes for histamine.

Patent document 1: Japanese Laid-Open patent publication (JP-A) No. H10-170514

Non patent document 1: Tetsuya Kondo, et al., (Aichi Industrial Technology Institute); the Proceeding of the 2001 Annual Meeting of the Japan Society for Bioscience, Biotechnology, and Agrochemistry (JBBA), P322 (2001).

### DISCLOSURE OF THE INVENTION

However, the conventional well-known diagnostic methods have required inspections to be conducted in hospitals or inspection agencies, that is, have required much labor. Moreover, there has been a problem that it takes much time to obtain inspection results. Thereby, there has been a problem that the symptoms are not improved because it is impossible at an early stage to determine whether the symptoms are caused by pollinosis or the cold, and to select an appropriate medicine. Furthermore, there has been a problem that it cost a great deal to specify an antigen causing the allergy.

Conventionally, there has been known a method in which the amount of pollens as an allergen is measured by counting the number of pollens adhered to slide glasses and the like, which are arranged at a predetermined position. However, such a method has a problem that the amount of pollens can be obtained only in areas in which the slide glasses and the like are arranged. Moreover, even when it is predicted that there is a small amount of pollens in a region, there is a case in which pollens have accumulated in the interior of a room in a building in the region to cause the symptoms of pollinosis.

The present invention has been made, considering the above circumstances, and an object of the invention is to provide a system supporting a diagnosis by which a morbidity possibility of a disease, the developing degree of which depends on positions and times, is easily judged. Another object of the invention is to provide a system by which the existing state of sufferers of the disease, the existing state of substances causing the disease, or, predictions of the above existing states are delivered or presented.

More particularly, the following services is provided: (1) a service helping a user distinguish pollinosis form other diseases at a current location in which the user stands; (2) a service helping a user to get relating information on a possibility of developing the disease and on the existing amount of a substance causing the disease with map information; and (3) a service sending warning information to a user suffering from pollinosis when the user is about to go to a region with a high possibility of developing pollinosis.

According to the present invention, there is provided a diagnostic support system which includes a mobile terminal and an analysis center which are connected to each other through a network, and judges a morbidity possibility that a user holding the mobile terminal is suffering from a disease. The mobile terminal includes: a detection unit which detects whether a feature component representing the feature of the morbidity of the disease is included in a sample collected from the user or not; and a transmission processing unit which transmits the detected result by the detection unit to the analysis center as symptom data representing the symptom of the user. The analysis center includes: a data obtaining unit which obtains the symptom data in correspondence with the position of the mobile terminal at which the symptom data was transmitted from the mobile terminal; a morbidity possibility calculation unit which calculates the morbidity possibility that the user is suffering from the disease, based on the symptom data and a reference parameter representing a feature caused in a sufferer of the disease; and an estimation processing unit which estimates the existing state of sufferers of the disease for each area, based on the morbidity possibility of a plurality of the users and corresponding the position. Here, the area means a zone having a predetermined range. The area can be defined, for example, by setting x-axes and a y-axes on a map. Here, the analysis center may further include a delivery unit which delivers the morbidity possibility and the existing state to the mobile terminal.

Here, the disease means a disease the developing degree of which depends on positions and times. Such a disease may include, for example, an allergic disease such as pollinosis; a disease caused by a cause locally generated, for example, by noises, unpleasant odors, and photochemical smog; an infectious disease such as influenza and a severe acute respiratory syndrome (SARS); and the like. The user using a mobile terminal means a user holding the mobile terminal.

The reference parameter may include data representing the feature of a sufferer, and data representing the feature of non-sufferer. The morbidity possibility calculation unit may calculate a morbidity possibility by comparison between symptom data and such a reference parameter or a standard value calculated based on the reference parameter.

Moreover, the morbidity possibility calculation unit may calculate the morbidity possibility by using various kinds of mathematical models. There may be possible, for example, a configuration in which a neural network (refer to, for example, Handbook of Neural Computation, Part C, Fiesler, E. and Beale, R. eds., Institute of physics publishing (Bristol) and Oxford University Press (New York), 1997) is made, based on a data set of the symptom data and the diagnosis results by doctors, and the symptom data transmitted from a user is input to the network to obtain a calculated result for the morbidity possibility. In this case, a position and a date can be transmitted from the mobile terminal, together with the symptom data, a new neural net work is made, using the above data as an input parameter, and, considering the position and the date, the morbidity possibility can be also calculated.

Moreover, the morbidity possibility calculation unit can calculate the morbidity possibility by cluster classification as a kind of multivariate analysis (refer to, for example, Tadaaki Miyamoto: “Introduction to Cluster Analysis”, published by Morikita Shuppan Co., Ltd., 1999). In this case, the reference parameters may be assumed to be a representative data group. Furthermore, the morbidity possibility calculation unit can calculate the morbidity possibility, using a decision-tree classification method such as ID3 (refer to, for example, C4.5—Programs for machine learning—, J. Ross Quinlan ed., Morgan Kaufmann publishers, 1993). In this case, the reference parameters can be assumed to be a classification rule. Moreover, when a user using the diagnostic support system of the invention for judgment of the morbidity possibility has diagnosis by a doctor, the diagnosis result can be incorporated into the reference parameters.

According to the diagnostic support system of the invention, the existing state of sufferers of a certain disease can be estimated for each area, based on the morbidity possibility of suffering from the disease. The diagnostic support system can deliver the existing state of such sufferers through a network and the like. Thereby, a number of people can obtain the developing state of a certain disease for each area; the developing state can be effectively used for prevention of the disease; and comparison between the symptom of a user and the developing state of another person can be utilized for determination of the morbidity possibility of the user.

In the diagnostic support system of the invention, the estimation processing unit can estimate the existing states of a causative substance causing a disease for each area, based on the existing states of sufferers of a disease. According to the above-described configuration, the existing state of a causative substance can be accurately estimated for each area, which is divided into areas with a smaller size. The diagnostic support system can deliver the existing state of such a causative substance through a network and the like. Thereby, a number of people can obtain the existing state of the causative substance causing a certain disease for each area, and comparison between the symptom of a user and the existing state of the causative substance can be utilized for determination of the morbidity possibility of the user.

In the diagnostic support system of the invention, the analysis center may further include a map-information storage unit which stores map information including information on buildings, and the estimation processing unit may estimate the existing state of sufferers of the disease for each area defined by each building, based on the morbidity possibility of the plurality of users, corresponding the position, and information on a building included in the map information.

In the diagnostic support system of the invention, the data obtaining unit may obtain the symptom data also in correspondence with a date on which the symptom data was made, and the estimation processing unit may estimate the existing state for each area and each period, based on the morbidity possibility for the plurality of users, and corresponding the position and the date.

Here, “a date on which symptom data was made” can be assumed to be, for example, a date on which a user collected the body fluids and the like of the user as a sample, a date on which a user detected the existence of a feature component by the detection unit, a date on which a user transmitted the symptom data to the analysis center, or a date oh which the analysis center received the symptom data.

In the diagnostic support system of the invention, the developing state of the disease and the existing state of the causative substance can be estimated for each area and each period, based on the morbidity possibility of suffering from a certain disease. The diagnostic support system can predict the developing state of a disease and the existing state of the causative substance, based on the developing states of the disease and the existing states of the causative substance for each area and each period. Moreover, the diagnostic support system can deliver such predictions through a network and the like. Thereby, a number of persons can act under a state in which measures against the disease is taken according to the predictions.

In the diagnostic support system of the invention, the analysis center may further include: a correction processing unit which corrects the morbidity possibility according to the existing state in an area including corresponding the position and in a period including the date; and a delivery processing unit which delivers the morbidity possibility corrected by the correction processing unit to the mobile terminal.

In the diagnostic support system of the invention, the analysis center may further include a delivery processing unit which delivers the morbidity possibility calculated based on the symptom data, together with the existing state in an area including corresponding the position and in a period including the date, to the mobile terminal.

In the diagnostic support system of the description of the invention, the mobile terminal may further include: a receiving unit which receives the morbidity possibility and the existing state in an area including corresponding the position; and a correction processing unit which corrects the morbidity possibility according to the existing state.

According to the present invention, there is provided a diagnostic support system which judges the morbidity possibility of a disease, comprising: a data obtaining unit which obtains symptom data representing the symptom of a test subject in correspondence with a position at which and date on which the symptom data was made; a morbidity possibility calculation unit which calculates a morbidity possibility that the test subject is suffering from the disease, based on the symptom data and reference parameters representing the features generated in sufferers of the disease; an estimation processing unit which estimates the existing state of sufferers of the disease for each area and each period; and a correction processing unit which corrects the morbidity possibility according to the existing state at the position and on the date.

A test subject can transmit data from a mobile terminal or a fixed terminal to the diagnostic support system. Here, “position at which the symptom data was made” can be assumed to be, for example, a position at which the symptom was caused to the test subject. When the test subject transmits the symptom data from the mobile terminal, the position of the mobile terminal, at which the test subject transmitted the symptom data to the analysis center, can be assumed to be “position at which the symptom data was made”. “Date on which the symptom data was made” can be assumed to be, for example, a date on which the symptom was caused to the test subject.

The estimation processing unit can estimate, for each area and each period, the existing state of the causative substance causing a disease, based on the existing state of sufferers of the disease.

The diagnostic support system according to the present invention may further include a map-information storage unit which stores map information including information on buildings, wherein the estimation processing unit may estimate the existing state of sufferers of the disease for each area defined by each building, based on the morbidity possibilities of the plurality of users, corresponding the positions, and information on buildings included in the map information.

The diagnostic support system according to the present invention may further include: a display processing unit which displays the existing state estimated by the estimation processing unit, together with the map information; and a selection accepting unit which accepts selection of a point included in map information displayed by the display processing unit from a user, wherein the display processing unit may display the existing state of sufferers at a point selected by the user in correspondence with a date. For example, when a user clicks a building on the map, the display processing unit can display the existing states of sufferers of a disease, and those of the causative substance for each area in the building, together with the building.

In the diagnostic support system of the invention, the map information may include information on each room in buildings, the estimation processing unit may estimate the existing states of sufferers of the disease for each area defined by each room, based on the morbidity possibilities of the plurality of users, corresponding the positions, and information on each room in buildings included in the map information, the diagnostic support system may further include: a display processing unit which displays the existing state estimated by the estimation processing unit, together with buildings included in the map information; and a selection accepting unit which accepts selection of a point defined by each of the rooms included in map information from a user, and the display processing unit may display the existing state of sufferers in the room selected by the user. Here, the display processing unit can display a building in two dimensions, or in three dimensions.

In the diagnostic support system of the invention, the data obtaining unit can obtain data indicating whether a feature component representing the feature of the morbidity of a disease exists in a sample collected from the test subject or not, and the morbidity possibility calculation unit can calculate the morbidity possibility that the test subject is suffering from the disease, based on the data indicating whether the feature component exists or not, and on the reference parameters. In this case, the terminal at the side of the test subject is provided with a detection unit which detects whether a feature component representing the feature of the morbidity of the disease is included in a sample collected from the user or not. Here, “a date on which symptom data was made” can be assumed to be, for example, a date on which a test subject collected the body fluids and the like of the test subject, a date on which a test subject detected the existence of a feature component by the detection unit, a date on which a test subject transmitted the symptom data to the analysis center, or a date on which the analysis center received the symptom data.

In the diagnostic support system of the invention, the data obtaining unit may obtain pieces of the symptom data in the same area and in the same period from a plurality of test subjects, the morbidity possibility calculation unit may calculate the morbidity possibilities for each of the pieces of symptom data of the plurality of test subjects, and the existing state obtaining unit may estimate the existing states for the area and the period, based on the morbidity possibilities of the plurality of test subjects.

In the diagnostic support system of the invention, the data obtaining unit may obtain a plurality of pieces of the symptom data in different areas, or different periods from the test subjects, the morbidity possibility calculation unit may calculate the morbidity possibilities for each of the plurality of pieces of symptom data, and the correction processing unit may correct the morbidity possibilities, based on relations between the plurality of morbidity possibilities and the plurality of existing states for respectively corresponding the areas and the periods.

In the diagnostic support system of the invention, the data obtaining unit may obtain information on whether the test subject develops a similar symptom to that developed at the time when the test subject collected the sample or not, in an area different from the area in which the data indicating whether the feature component exists or not was acquired or a period different from the period in which the data indicating whether the feature component exists or not was acquired, the morbidity possibility calculation unit may calculate the morbidity possibility of the test subject for the time at which the information was acquired from the test subject, based on the data indicating whether the feature component exists or not, and on the information, and the correction processing unit may correct the morbidity possibility, based on relations between the plurality of morbidity possibilities and the plurality of existing states for the causative substance in the areas and the periods, which are corresponding to the possibilities.

According to the present invention, there is provided a diagnostic support system calculating the morbidity possibility of a disease, including: a data obtaining unit which obtains symptom data representing the symptom of a test subject in correspondence with the position at which and with the date on which the symptom data was made; a morbidity possibility calculation unit which calculates a morbidity possibility that the test subject is suffering from the disease, based on the symptom data and reference parameters representing a feature which is generated in sufferers of the disease; and an estimation processing unit which predicts the existing state of sufferers of the disease for each area or each period, based on the morbidity possibilities calculated the plurality of pieces of symptom data in different areas or different periods.

According to the present invention, there is provided a mobile terminal which is used for a diagnostic support system provided with an analysis center which judges a morbidity possibility that a user is suffering from a disease, based on symptom data representing the symptom of the user, including: a detection unit which detects whether a feature component representing the feature of the morbidity of the disease exists in a sample collected from the user or not; and a transmission processing unit which transmits the detected result by the detection unit to the analysis center as symptom data representing the symptom of the user.

The mobile terminal according to the present invention may further include: a receiving unit-which receives the morbidity possibility judged based-on the symptom data and the existing state of a causative substance causing the disease in a position at which and on a data on which the sample was collected; and a correction processing unit which corrects the morbidity possibility according to the existing state.

The mobile terminal according to the present invention may further include: an input accepting unit which accepts, from a user, input of information on whether the user develops a similar symptom to that developed at the time when the user collected the sample or not, in an area different from the area where the user collected the sample or a period different from the period in which the user collected the sample, and a morbidity possibility calculation unit which calculates the morbidity possibility of the user for a time at which the information was obtained from the user, based on the morbidity possibility determined based on the symptom data, and on the information, wherein the receiving unit may also receive the existing state in a position at which and on a date on which said user input said information, and said correction processing unit may correct said morbidity possibilities, based on relations between said plurality of morbidity possibilities and said plurality of existing states at said positions and on said dates respectively corresponding to said possibilities.

The present invention provides a system supporting a diagnosis by which a morbidity possibility of a disease, the developing degree of which depends on positions and times, is easily judged.

### BRIEF DESCRIPTION OF THE DRAWINGS

The above-described and the other objects, features and advantages will become further clear from the following description of the preferred embodiments taken in conjunction with the accompanying drawings.

FIG. 1 is a view showing a diagnostic support system including a mobile terminal and an analysis center according to an embodiment of the present invention;

FIG. 2 is a block diagram showing a configuration of the mobile terminal and the analysis center according to the embodiment of the invention;

FIG. 3 is a view showing one example of a chip according to the embodiment of the invention;

FIG. 4 is a view showing one example of the mobile terminal according to the embodiment of the invention;

FIG. 5 is a sectional view taken along the C-C′ line in FIG. 4(a);

FIG. 6 is a view showing one example of a data structure for an analysis-information storage unit shown in FIG. 2;

FIG. 7 is a view showing one example of a data structure for a data storage unit shown in FIG. 2;

FIG. 8 is a view showing one example of a data structure for an estimation-result storage unit shown in FIG. 2;

FIG. 9 is a view showing one example of a data structure for a user-information storage unit shown in FIG. 2;

FIG. 10 is a view showing morbidity possibilities of a plurality of users in a predetermined area and a predetermined period in a statistical manner;

FIG. 11 is a view showing one example of a data structure for an area-information storage unit shown in FIG. 2;

FIG. 12 is a view showing relations between the morbidity possibility calculated by the morbidity possibility calculation unit, based on the symptom data for a certain user and the existing states of the causative substance causing pollinosis on the corresponding dates and at the corresponding positions;

FIG. 13 is a flowchart showing processing procedures in the mobile terminal and the analysis center according to the embodiment of the present invention;

FIG. 14 is a block diagram showing a configuration for a mobile terminal 14 according to an embodiment of the present invention;

FIG. 15 is a view showing one example of a questionnaire sheet;

FIG. 16 is a view showing a screen, which displays the existing state of a causative substance, which has been delivered from a estimation processing unit, together with map information;

FIG. 17 is a view showing a structure of a sample separation unit in the chip;

FIG. 18 is a view showing another example for the chip shown in FIG. 3;

FIG. 19 is a view showing another example for the chip shown in FIG. 3;

FIG. 20 is a view showing a connector, which connects the chip, which has been explained, referring to FIG. 19, and an external light source and an external detector; and

FIG. 21 is a view showing a change over time of existing state of the causative substance for the position in the map information shown in FIG. 16.

### BEST MODE FOR CARRYING OUT THE INVENTION

### First Embodiment

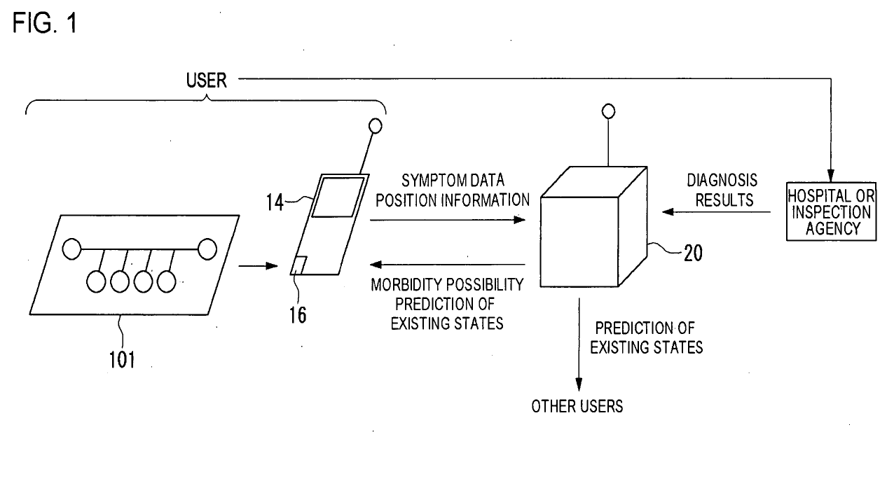

FIG. 1 is a view showing a diagnostic support system including a mobile terminal 14 and an analysis center 20 according to a first embodiment of the invention. In this embodiment, the diagnostic support system judges whether a user is suffering from a pollinosis or not.

A user using the mobile terminal 14 transmits symptom data representing his or her symptoms from the mobile terminal 14 to the analysis center 20, wherein the symptom data is used for determining whether the user is suffering from pollinosis or not. In this embodiment, the symptom data is data that indicates whether or not a feature component representing the feature of pollinosis is included in samples, such as nasal mucus and blood, which are collected from a user. Here, a chip 101 containing a coloring agent and the like which forms a color by existence of such a feature component is used. The feature component may include, for example, histamine, IgE, or leukotriene. The configuration of the chip 101 will be described later.

The mobile terminal 14 is a mobile phone, a PDA (Personal Digital Assistant), and the like with a communication function. The mobile terminal 14 includes a detection unit 16, which detects whether the color of the coloring agent in the chip 101 is formed. Moreover, the mobile terminal 14 has a configuration in which determined results transmitted from the analysis center 20 can be presented to the user.

In this embodiment, a user who wants to know whether he or she is suffering from pollinosis is required to buy the chip 101 beforehand. The user collects his or her body fluids such as nasal mucus and blood, and the body fluids are introduced as a sample into the chip 101 to be reacted with the coloring agent. Thereafter, the detection unit 16 in the mobile terminal 14 detects the formed color of the coloring agent in the chip 110. The mobile terminal 14 transmits the detected result as symptom data representing the symptom of the user to the analysis center 20. It is assumed in this embodiment that processes from a step, at which the user collects his or her body fluids, to a step, at which the symptom data is transmitted to the analysis center 20, are continuously executed at a same location within a predetermined time.

The analysis center 20 judges the morbidity possibility that the user is suffering from pollinosis, based on the symptom data transmitted from the user and on reference parameters representing symptom features of sufferers of pollinosis, to transmit the judged morbidity possibility to the mobile terminal 14. Thereby, only with a simple method, the user of the mobile terminal 14 can know, without going to a hospital, an inspection agency, or the like, the morbidity possibility that the user himself or herself is suffering from pollinosis.

Moreover, the analysis center 20 obtains information on the position of the mobile terminal 14, at which the user has transmitted the symptom from the mobile terminal 14. In this embodiment, the position of the mobile terminal 14, at which the user has transmitted the symptom data from the mobile terminal 14, can be assumed, for processing, to be a position at which the user develops the symptom because processes from a step, at which the user collects the his or her body fluids, to a step, at which the symptom data is transmitted to the analysis center 20, are continuously executed as described above. The analysis center 20 estimates the existing state of sufferers of pollinosis, and that of the substance causing the disease, based on pieces of the symptom data of a plurality of users and information on positions of mobile terminals 14 at which the pieces of the symptom data have been transmitted. According to the existing state of such sufferers or that of the causative substance, the analysis center 20 can correct the morbidity possibility that each of the users suffers from the disease. Furthermore, the analysis center 20 can deliver the existing state of sufferers of the disease and that of the causative substance for each area to a plurality of users, or can disclose the above states on a web page and the like through a network. Here, the size of an area can be arbitrarily specified according to a desired range of a user. The area can be assumed to be, for example, a local region, or a region, a building, or a room of the building specified by a geographic name. The analysis center 20 can deliver or disclose the existing state of sufferers of the disease and that of the causative substance for each area by mapping them on a map. Moreover, when a user has a checkup for a diagnosis of a doctor or a through more detailed medical examination in a hospital or an inspection agency, the analysis center 20 can obtain data representing the result of the diagnosis or the examination, and can incorporate the result into the reference parameters for determination of the morbidity possibility.

Moreover, the analysis center 20 can obtain information for specifying a causative substance, by which a symptom is developed, from the user together with the symptom data. A causative substance can be specified, by using a chip in which a plurality of allergens which may cause pollinosis are introduced into different liquid holders, respectively. The user introduces his or her body fluids as a sample into the chip with the above-described configuration for reaction of the sample with various kinds of allergens. By detecting whether an antibody (IgE) combining with a specific antigen exists in the body fluids of the user or not, it can be specified whether the user have an allergic disease for any one of the allergens or not. An enzyme-linked immunosorbent assay (ELISA) can be used for the above processing. In this method, the user introduces samples into a chip in the first place, and, after the samples are reacted with allergens, unreacted samples are washed and removed. Subsequently, second antibodies adhered to antibodies (first antibodies) such as IgE are introduced into the chip for reaction with the samples, and unreacted second antibodies are washed and removed. Enzymes are combined with the second antibodies. Then, a color forming material, which forms a color by deconfiguration through the enzymes combined with the second antibodies is introduced into the chip. Thus, by bringing the kinds of the allergens introduced into the liquid holders into correspondence with the degrees of forming a color, the user can determine to what kind of allergen the user has an allergic disease because the color forming materials form a color in a liquid holder in which the antibody such as IgE is generated. When the user transmits the detection results obtained by using this chip, and the detection results obtained by use of the above-described chip 101 from the mobile terminal 14 to the analysis center 20, the existing state of sufferers of the disease and that of the causative substance can be estimated for each causative substance in the analysis center 20.

It depends on the existing amount of the causative substance (pollen) whether an allergic disease such as pollinosis is developed or not. Accordingly, sufferers of pollinosis repeat development and remission of the allergy many times in the season in which the pollens are scattering, and the sufferers are required to determine whether the symptoms are caused by the allergy or by other diseases such as the cold, whenever the sufferers develop the symptom. Moreover, it depends on the constitution of the sufferer of the allergic disease such as pollinosis whether the allergic disease is developed or not, even when the existing amount of the causative substance such as pollens is the same. In this case, a user who has been diagnosed as having pollinosis in a hospital or the like can more definitely estimate that the symptom is caused by pollinosis, in comparison with a user who has not been diagnosed as having pollinosis, even when the both users have a similar symptom. A diagnosis can be made more accurately for each user by “correcting judgment standards dedicatedly for an individual” using diagnosis results obtained for the user himself or herself in a hospital.

Moreover, the developing degree of the symptoms depends on the scattering amount of pollens even for a user who has been diagnosed as having pollinosis in a hospital or the like. Thereby, inspections using the chip 101 are executed at different times and places, and the inspected results are received from the analysis center 20 at each inspection, and measures against pollens can be taken.

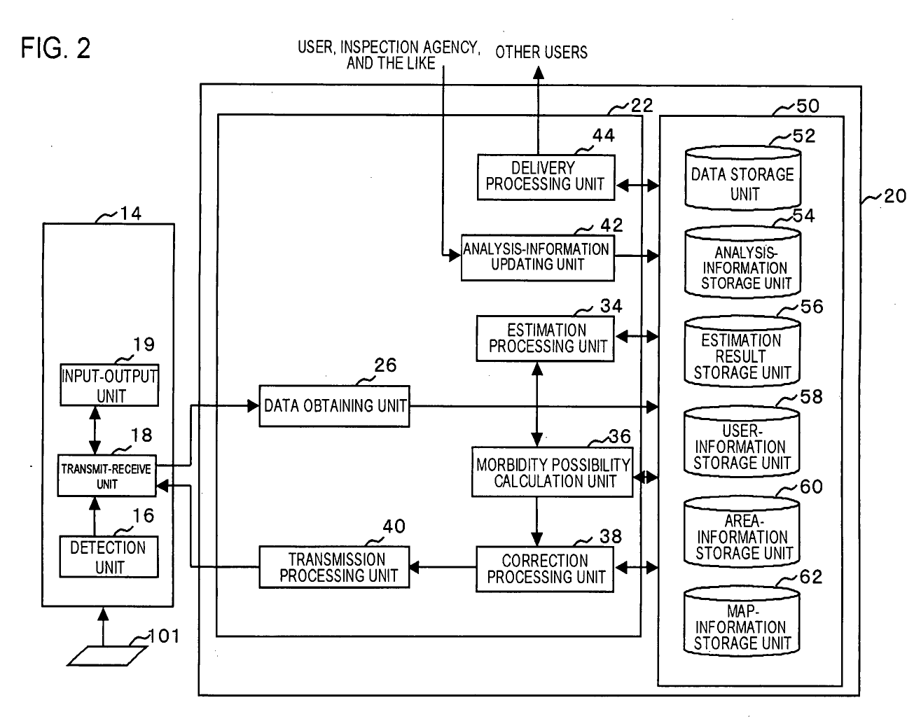

FIG. 2 is a block diagram showing a configuration of the mobile terminal 14 and the analysis center 20 according to this embodiment.

The analysis center 20 includes an analysis processing unit 22 and a database 50. The analysis processing unit 22 includes: a data-obtaining unit 26; an estimation processing unit 34; a morbidity possibility calculation unit 36; a correction processing unit 38; a transmission processing unit 40; an analysis-information updating unit 42; and a delivery processing unit 44.

The database 50 includes: a data storage unit 52; an analysis-information storage unit 54; an estimation-result storage unit 56; a user-information storage unit 58; an area-information storage unit 60; and a map-information storage unit 62.

Components, expressed as hardware components, of the analysis center 20, are realized mainly by a CPU, memories, programs which are loaded in the memories and actualize the components illustrated in the drawing, a storage unit, such as a hard disk, which stores the programs, and an interface for connection to a network in an computer and those skilled in the art understands that various modifications and applications may be applied as a method and a device. Drawings, which will be explained below, will show functional blocks instead of showing hardware units.

The data-obtaining unit 26 obtains symptom data from the mobile terminal 14. The data-obtaining unit 26 may acquire symptom data of a user in correspondence with the position at which the symptom data was made, and the date on which the symptom data was made. “Date at which symptom data is made” maybe assumed to be a date, for example, on which a user collected his or her body fluids, on which a user detected a feature using the chip 101, on which a user detected that the chip 101 formed the color using the mobile terminal 14, or on which a user transmitted symptom data from the mobile terminal 14. Moreover, “date at which symptom data is made” may be also assumed to be a date on which the analysis center 20 acquired symptom data. Such a date may be determined, based on a timer function of the mobile terminal 14, or on that of the analysis center 20, or may be determined by input by the user.

“Position at which symptom data is made” may be assumed to be, for example, information on a position of the mobile terminal 14 where the user using the mobile terminal 14 transmits symptom data to the analysis center 20. “Information on a position of the mobile terminal 14” may be obtained by using a position detecting function of a base station in a mobile phone network through the use of a radio receiving state of the mobile terminal 14. Moreover, when the user holds the mobile terminal 14 with the Global Positioning System (GPS) function, “information on a position of the mobile terminal 14” may be acquired by using the GPS function. Furthermore, the user is configured to input information on a position, at which he or she stands, from the mobile terminal 14. The information on the position of the mobile terminal 14 is transmitted to the analysis center 20 together with the symptom data. The information on a position may be configured to be not only two-dimensional information, but also three-dimensional one including the height.

When the data-obtaining unit 26 obtains symptom data from the mobile terminal 14, a questionnaire sheet for making questions on a user's anamnesis maybe also transmitted to the user using the mobile terminal 14, wherein the user inputs his or her subjective symptoms. The data-obtaining unit 26 may also obtain answers, which are input by the user through the mobile terminal 14, to the questionnaire sheet as the symptom data.

The data-obtaining unit 26 writes the symptom data into the data storage unit 52 in correspondence with the position at which the symptom data was made, and the date on which the symptom data was made.

The morbidity possibility calculation unit 36 calculates a morbidity possibility that the user is suffering from pollinosis, based on the symptom data and the reference parameter. The analysis-information storage unit 54 stores the reference parameters to which the morbidity possibility calculation unit 36 refers for calculation of the morbidity possibility. The analysis-information storage unit 54 may store the questionnaire sheet transmitted by the data-obtaining unit 26 as well. In correspondence with the symptom data, the data storage unit 52 stores the morbidity possibility that the user suffers from pollinosis, wherein the morbidity possibility calculation unit 36 has calculated the morbidity possibility, based on the symptom data.

The estimation processing unit 34 estimates the existing state of sufferers of pollinosis and that of the causative substance causing pollinosis for each area and each period, based on the morbidity possibilities, the positions, and the dates of a plurality of users, referring to the data storage unit 52. The analysis center 20 may include, for example, means for acquiring information on scattering causative substances which cause pollinosis, information on weather conditions and the like, and the estimation processing unit 34 may estimate the existing state of sufferers of pollinosis and that of the substance causing pollinosis, considering the above information as well. According to the diagnostic support system in this embodiment, the existing amount of the causative substance and the like can be obtained for smaller areas than those by the conventional method in which the amount of pollens as an allergen is measured by counting the number of pollens adhered to slide glasses and the like which are arranged at predetermined positions because the existing state of the causative substance is estimated, based on the symptom data transmitted form the users at various positions. Accordingly, the existing state of the causative substance can be more accurately estimated.

Furthermore, the estimation processing unit 34 may predict the existing state of sufferers of pollinosis, and that of the causative substance causing pollinosis. The estimation processing unit 34 predicts the existing state of sufferers of pollinosis, and that of the causative substance causing pollinosis, considering pieces of symptom data acquired from users, the dates on which the pieces of symptom data were made and the positions at which the pieces of symptom data were made, pieces of personal information such as diagnosis results by doctors for users, calculated results by the morbidity possibility calculation unit 36, based on the symptom data, and measured outside information such as information on scattering causative substance which causes pollinosis, and information on weather conditions. The prediction by the estimation processing unit 34 can be executed by using various kinds of mathematical models. The estimation-result storage unit 56 stores the existing state of sufferers of pollinosis and that of the causative substance causing pollinosis, and predicted results thereof, estimated by the estimation processing unit 34, for each area and each period.

The delivery processing unit 44 processes delivery of the existing state of sufferers of pollinosis, that of the causative substance, and predicted results thereof for each area and each period to other users and the like.

The map-information storage unit 62 stores map information. The delivery processing unit 44 can deliver the existing state of sufferers of pollinosis and that of the causative substance together with the map information stored in the map-information storage unit 62. Thereby, other users can know the existing state of sufferers of pollinosis and that of the causative substance for a predetermined area and a predetermined period and can effectively utilize the states for prevention of pollinosis and for determination of him or her morbidity possibility.

Moreover, the map-information storage unit 62 may store information on buildings in the areas included in the map information, and the estimation processing unit 34 may estimate the existing state of sufferers of pollinosis and that of the causative substance causing pollinosis for each area in buildings. Furthermore, the map information may include information for each room in the buildings. Thereby, the existing state of sufferers of pollinosis and that of the causative substance causing the allergy can be estimated for further divided area. Moreover, the delivery processing unit 44 may also deliver such estimated results to, for example, building managers and the like. Thereby, when the diagnostic support system according to the invention is applied to, for example, diagnostic support of a disease caused by unpleasant odors and noises, the building managers can be urged to remove the cause of the disease.

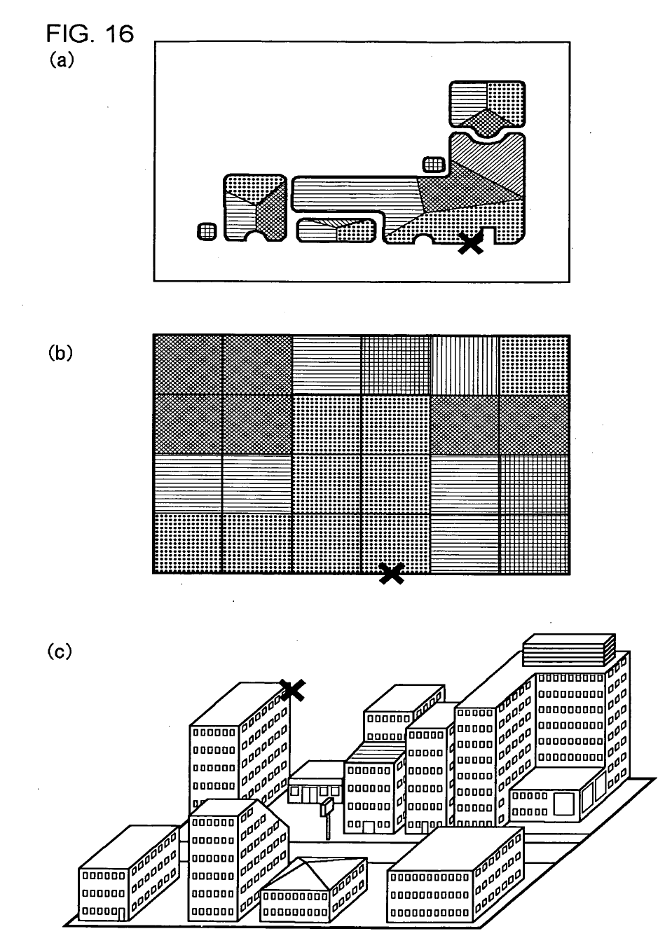

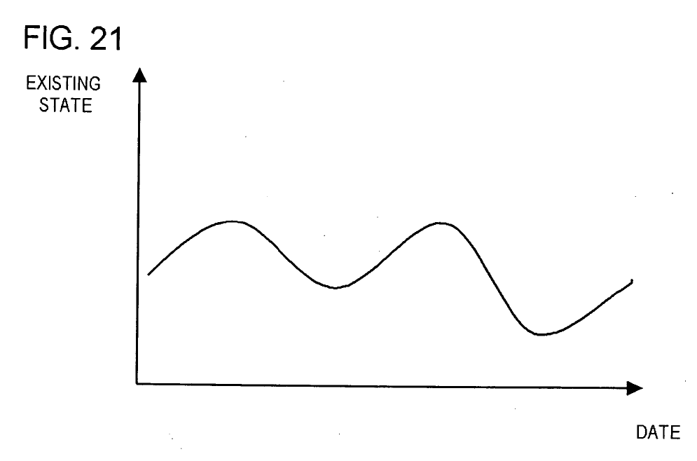

FIG. 16 is a view showing screens, which display the existing state of a causative substance delivered from the delivery processing unit 44, together with the map information. For example, when a user selects a region in the map information shown in FIG. 16(a), the delivery processing unit 44 may deliver data in such a way that the existing states of the causative substance are displayed, as shown in FIG. 16(b), for each area included in the region. Moreover, as shown in FIG. 16(c), the existing state of the causative substance may be delivered for each building, or the existing state of the causative substance for a specific floor or room of a building may be delivered. Furthermore, the delivery processing unit 44 may receive an input specifying a delivery position desired by a user, and may deliver data in such a way that the existing states of the causative substance are displayed in time sequence for the specified position. FIG. 21 is a view showing the change of an existing state of the causative substance over time for a selected position in a building wherein the selected position has been selected by a user in FIG. 16(c). The delivery processing unit 44 delivers data as shown in FIG. 21 to the user. Though the existing state of the causative substance is shown as ordinate in FIG. 21, the delivery processing unit 44 may deliver a diagram in which the morbidity possibilities for each user or the existing states of sufferers are shown as ordinate.

Returning back to FIG. 2, the user-information storage unit 58 stores user IDs, user mail-addresses, and the like for each user. The area-information storage unit 60 stores position information for a plurality of areas.

Referring to the data storage unit 52 and the estimation-result storage unit 56, the correction processing unit 38 corrects the morbidity possibility that a user suffering from pollinosis, considering the symptom data obtained from the user, and the existing state of sufferers of pollinosis, or the existing state of the causative substance for the date on which the symptom data was made and the position at which the symptom data was made. The correction processing unit 38 may correct the morbidity possibility that the user suffers of pollinosis, considering predicted results for the existing state of sufferers of pollinosis and that of the causative substance at the date on which the symptom data was obtained from the user.

The transmission processing unit 40 transmits the morbidity possibility calculated by the morbidity possibility calculation unit 36 and the morbidity possibility corrected by the correction processing unit 38 to the mobile terminal 14. The data-obtaining unit 26 receives the user ID from the user together with the symptom data. Based on the user ID, the transmission processing unit 40 transmits the morbidity possibility to the user's mail address, referring to the user-information storage unit 58.

The analysis-information updating unit 42 receives a diagnosis result by a doctor from the user using the mobile terminal 14, or from a hospital or an inspection agency, and, updates information on the reference parameters and the like stored in the analysis-information storage unit 54, based on the diagnosis result. Updating of the reference parameters will be described later.

The mobile terminal 14 includes the detection unit 16, a transmit-receive unit 18, and an input-output unit 19. Here, the detection unit 16 is, for example, a spectrophotometer, a fluorophotometer, a CCD camera, or the like. The transmit-receive unit 18 transmits detected results detected by the detection unit 16 to the analysis center 20 as symptom data representing the symptom of a user. Moreover, the transmit-receive unit 18 receives the morbidity possibility calculated in the morbidity possibility calculation unit 36, and the morbidity possibility corrected by the correction processing unit 38 from the analysis center 20. The transmit-receive unit 18 forwards the received morbidity possibility to the input-output unit 19. The input-output unit 19 outputs the morbidity possibility to the display unit and the like (not shown in the drawings) for presentation to a user.

Subsequently, a specific example of a configuration of the chip 101 will be explained.

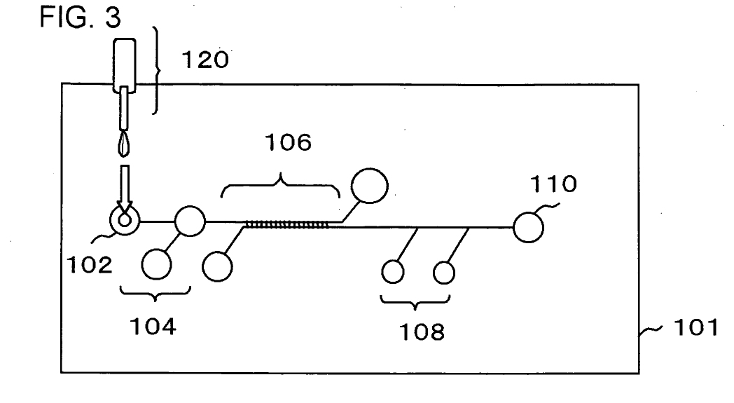

FIG. 3 is a view showing one example of the chip 101 according to this embodiment. In this embodiment, the chip 101 is used for detecting whether a feature component, such as histamine, IgE, or leukotriene, which represents the distinctive characteristic of pollinosis are included in the body fluids of a test subject or not, and, when included, for detecting what degree of the element is included. When, for example, histamine is detected, the ELISA, a fluoroscopy, or a method in which a sensor is used (non patent document 1) may be used. When a sensor is used, the sensor may be provided like the chip 101, or may be provided at the side of the mobile terminal 14. Moreover, when IgE is detected, an EIA or enzyme immunoassay (immunity measurement method) may be used. ORITON IgE “CHEMIPHAR” from Nippon Chemiphar Co., Ltd., may be listed as an example for detecting IgE by using the EIA. Moreover, when leukotriene is detected, the ELISA may be used.

The chip 101 is formed with a size capable for a user of carrying. Moreover, the chip 101 according to this embodiment is used in combination with a sampling apparatus 120, such as a cotton swab, a dropper, or an injection needle, which is used for collecting the body fluids of the user. The chip 101 includes: a sample introduction unit 102; a pretreatment unit 104; a sample separation unit 106; a detection and reaction unit 108; and a waste fluid holder 110. The chip 101 may be formed of, for example, plastic, and the sample introduction unit 102, the pretreatment unit 104, the sample separation unit 106, the detection and reaction unit 108, the waste fluid holder 110, and the like are provided by forming grooves and fluid holders on a plastic board. Moreover, a lid (not shown in the drawings) may be provided for the chip 101, and the sample introduction unit 102 and the waste fluid holder 110 may have an open configuration. A dried sample is set in the pretreatment unit 104 and the sample separation unit 106.

For example, lysozyme chloride is introduced into the pretreatment unit 104 as a viscosity reducing agent, and the viscosity of the sample can be reduced by mixing the sample with the lysozyme chloride when a sample is introduced from the sample introduction unit 102. Moreover, an appropriate buffer can be introduced into the pretreatment unit 104 to adjust the pH value of the sample. Furthermore, a filter may be provided in the pretreatment unit 104 to remove impurities. The sample is separated in the sample separation unit 106 to remove cells, and only a liquid element is introduced into the detection and reaction unit 108.

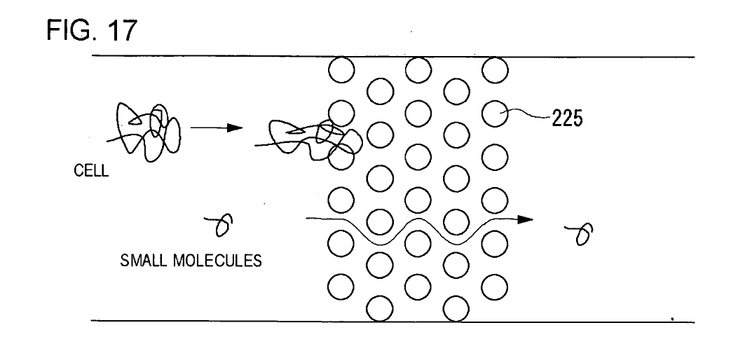

Subsequently, a structural example of the sample separation unit 106 in the chip 101 will be explained, referring to FIG. 17. FIG. 17 is a view showing the detailed structure of the sample separation unit 106 in FIG. 3. The chip 101 may be formed of, for example, silicon, glass, quartz, various kinds of plastic materials, or an elastic material such as rubber. The sample separation unit 106 is a filter formed of an obstacle with a clearance having a size (for example, 0.1 micrometers to 1 micrometers) in such a way that cells, or destroyed structures of the cells cannot pass through the clearance. The obstacles may be a forest of pillars, parallel walls, twisted yarns, and porous materials. Here, the sample separation unit 106 may be realized by groove portions provided in the above-described materials and column-like pillars 225 arranged in the groove portions. The sample passes through the clearances among the pillars 225 in the sample separation unit 106 with the above-described configuration. Here, the sample having molecules with the larger size is much more blocked by the pillars 225 to increase the time during which the sample passes through the sample separation unit 106. The sample having molecules with the smaller size relatively smoothly passes through the clearances between the pillars 225 to reduce the time during which the sample passes through the sample separation unit 106. Thereby, the cells can be removed to introduce only a liquid element into the detection and reaction unit 108.

Returning back to FIG. 3, a coloring agent, which forms a color according to the existence of a feature component is introduced into the detection and reaction unit 108. When the feature component is histamine, for example, a diazo coupling agent can be used as a coloring agent. Here, the detection and reaction unit 108 may be provided with a plurality of fluid holders, and one of the fluid holders, into which the coloring agent is not introduced, can be used as a reference fluid holder.

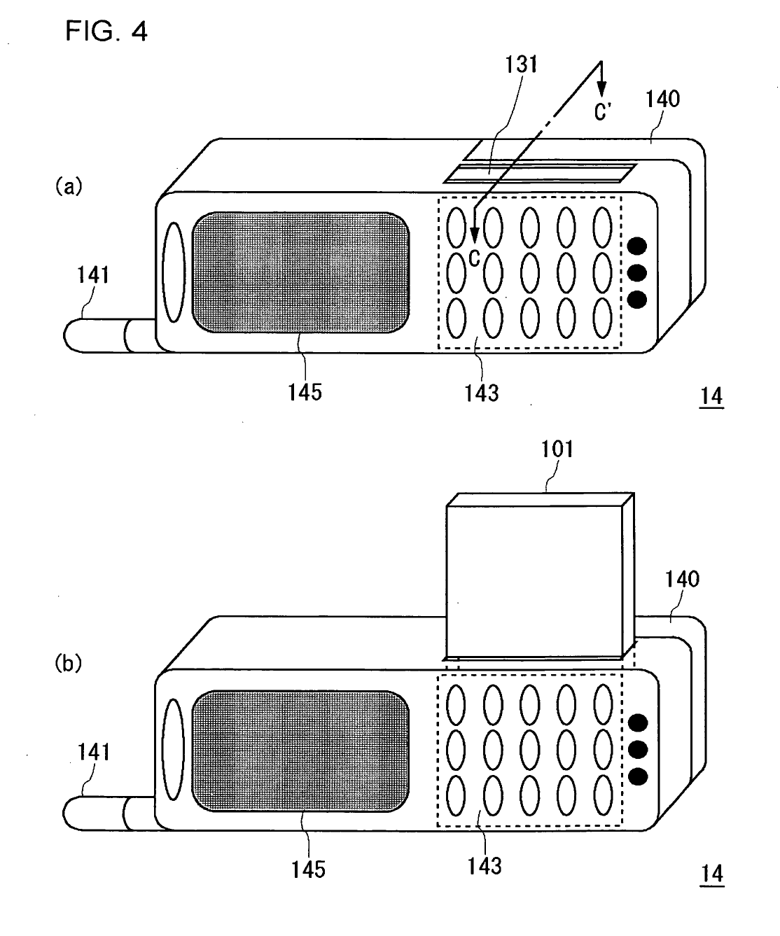

FIG. 4 is a view showing one example of the mobile terminal 14 according to this embodiment. Here, an example in which the detection unit 16 is assumed to be a spectrophotometer will be explained. The mobile terminal 14 is provided with a chip insertion unit 131 for inserting the chip 101. FIG. 4(a) shows a state in which the chip 101 is not inserted into the mobile terminal 14, and FIG. 4(b) shows a state in which the chip 101 is inserted into the mobile terminal 14. The mobile terminal 14 has a battery pack 140, an antenna 141, a functional button group 143, a display unit 145, and the like, like a mobile terminal such as an ordinary mobile phone.

Here, although the configuration in which the detection and reaction unit 108 is provided in the mobile terminal 14 the detection and reaction unit 108 may be formed as a device separated from the mobile terminal 14, and may be connected to the mobile terminal 14. Thereby the configuration of the mobile terminal 14 can be made simple, and the sample such as body fluids can be prevented from adhering to the mobile terminal 14. In this case, connection between the mobile terminal 14 and the detection and reaction unit 108 may be made by cable or by wireless. The connection may be made, for example, through a universal serial bus (USB) terminal, or by wireless communication means such as the Bluetooth communication. Preferably, a device including the detection and reaction unit 108 is made waterproof and washable.

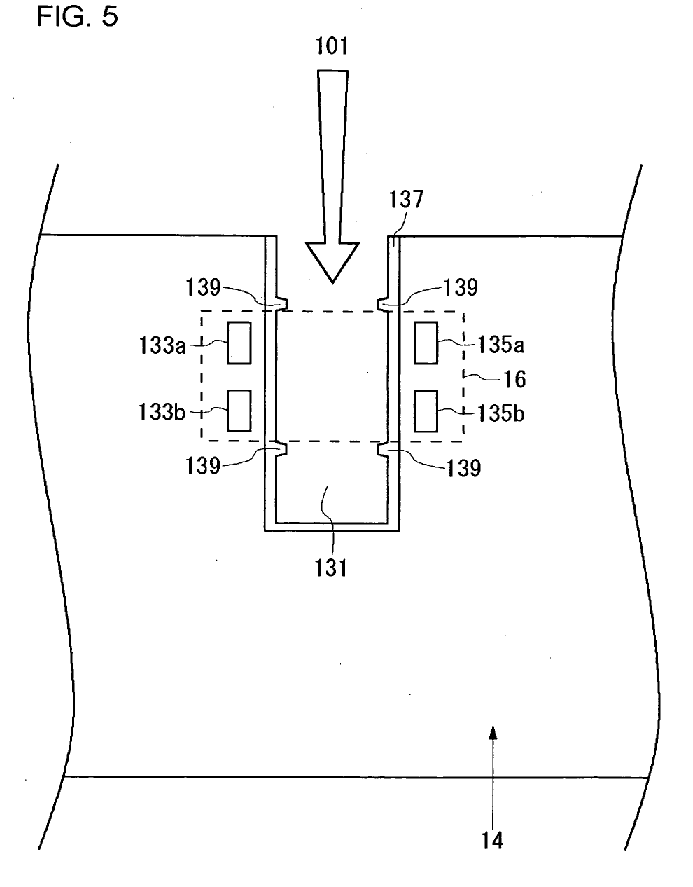

FIG. 5 is a sectional view taken along the C-C′ line in FIG. 4(a).

As shown in FIG. 5, the chip insertion unit 131 in the mobile terminal 14 is provided with the detection unit 16. The detection unit 16 includes a light source 133a and a light source 133b for light irradiation, and a light receiving unit 135a and a light receiving unit 135b respectively detects light from the light source 133a and the light source 133b. The light source 133a and the light source 133b are provided at a position in such a way that light can be irradiated onto the detection and reaction unit 108 in the chip 101 when the chip 101 is inserted into the chip insertion unit 131. The light receiving unit 135a and the light receiving unit 135b are provided in such a way that the units 135a and 135b can detect light, which has passed through the detection and reaction unit 108. One of the light source 133a and the light source 133b can be used for irradiating light onto the reference fluid holder. A gasket 137 formed with a convex portion 139 for holding the chip 101 is provided in the chip insertion unit 131 of the mobile terminal 14. Although not shown in the drawings, a concave portion in engagement with the convex portion 139 of the gasket 137 may be provided in the chip 101, and the chip 101 can surely be installed in the chip insertion unit 131 by the above engagement. Thereby, the light from the light source 133a and the light source 133b is securely irradiated onto the detection and reaction unit 108 in the chip 101, and the light which passes through the detection and reaction unit 108 is surely received by the light receiving unit 135a and the light receiving unit 135b.

The light receiving unit 135a and the light receiving unit 135b convert the strength of the received light to a current (a current value or a voltage value). The detection unit 16 includes an operation unit (not shown in the drawings) by which the transmittance is calculated, based on the current values, which are obtained through conversion by the light receiving unit 135a and the light receiving unit 135b. The light source 133a and the light source 133b may be assumed to be, for example, a light emitting diode. Moreover, the light receiving unit 135a and the light receiving unit 135b may be assumed to be, for example, a phototransistor. Here, although not shown in the drawings, the mobile terminal 14 may have a spectroscopic unit by which the light irradiated from the light source 133a and the light source 133b is separated into the spectral components through an optical filter to irradiate a light with a predetermined wavelength. According to the above configuration, the existing amount of a feature component with a peak at a specific wavelength can be detected.

The mobile terminal 14 can store the date on which the chip 101 was inserted into the chip insertion unit 131, or the date on which the detection unit 16 detects that the detection and reaction unit 108 in the chip 101 forms a color in correspondence with the detected result. The transmit-receive unit 18 (See FIG. 2) may transmit these dates as a date on which the symptom data was made to the analysis center 20. The transmit-receive unit 18 transmits the transmittance detected by the detection unit 16 as the symptom data to the analysis center 20, based on an instruction from a user. Here, the transmit-receive unit 18 may transmit the symptom data in any form, for example, the transmittance can be quantized in the mobile terminal 14 to the analysis center 20. Thereby, the traffic amount of the data from the mobile terminal 14 to the analysis center 20 can be reduced to save the communication charge. Thus, the objectivity of the symptom data for a user can be ensured by transmitting the results detected in the detection unit 16 to the analysis center 20.

Here, although it is described that the detection unit 16 detects the transmittance, the detection unit 16 may be also configured to detect the absorbance or the scattering characteristic.

Moreover, the configuration of the chip 101, and that of the detection unit 16 in the mobile terminal 14 are not limited to the above-described ones, and various kinds of modifications may be possible.

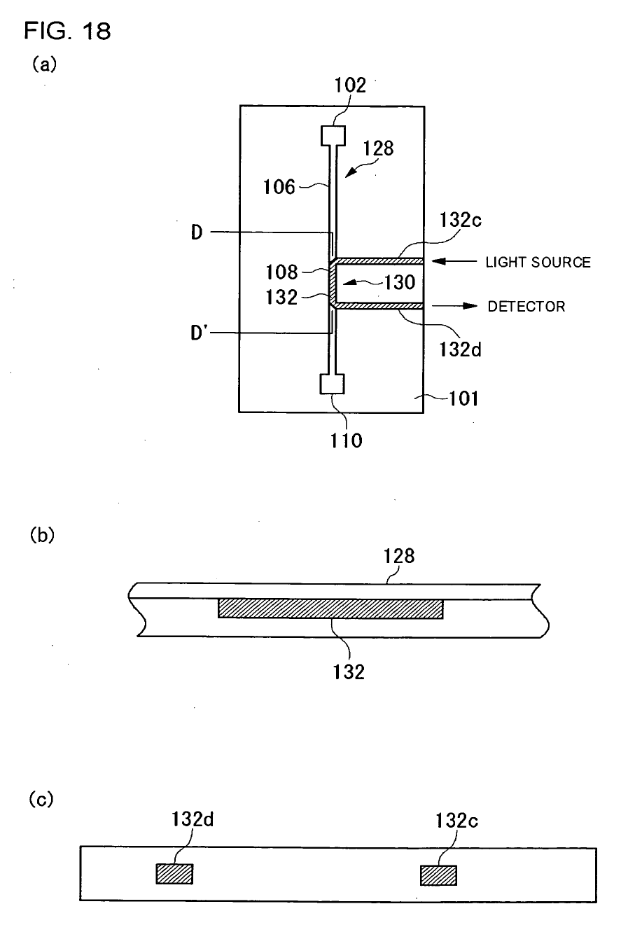

For example, as shown in FIG. 18(a), the sample separation unit 106 and the detection and reaction unit 108 may be provided on a channel 128, and an optical waveguide 132 may be formed under the detection and reaction unit 108. Here, the optical waveguide 132 may be formed of, for example, a quartz material, or a polymer organic material. The optical waveguide 132 is configured to have a higher refractive index than those of surrounding materials. In this case, light is introduced from the side of the chip 101 into the optical waveguide 132, and, similarly, light is derived from the side of the chip 101. FIG. 18(b) is a sectional view taken along the D-D′ line in FIG. 18(a). FIG. 18(c) is a side view showing the optical waveguide 132c for light irradiation, and the optical waveguide 132d for light receiving, shown in FIG. 18A. In this case for example, alight source by which light is introduced into the optical waveguide 132c for light irradiation of the chip 101, and a detector receiving light from the optical waveguide 132d for light receiving can be provided on the sidewall, the bottom, or the like of the mobile terminal 14. According to the above-described configuration, introduction of light into the detection and reaction unit 108 and detection of light from the detection and reaction unit 108 can be performed by contacting the exposed surface of the optical waveguide 132c for light irradiation and the optical waveguide 132d for light receiving in the chip 101 with the sidewall or the bottom, or the like of the mobile terminal 14.

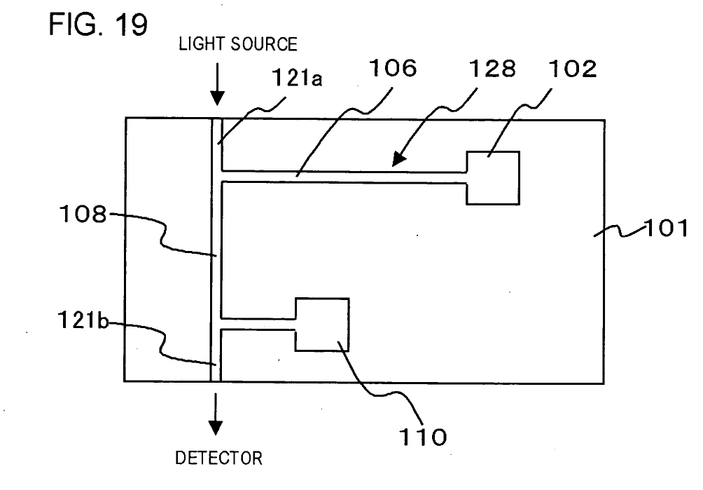

Furthermore, the chip 101 may have a configuration shown in FIG. 19. Even in this case, the detection and reaction unit 108 is provided on the channel 128. Here, the chip 101 may be formed of a metallic material or a material with a lower refractive index than that of the sample in a region in which at least the detection and reaction unit 108 is provided. According to the above-described configuration, the light introduced from the light introduction unit 121a into the channel 128 can be configured to be forwarded along the detection and reaction unit 108 with the light being trapped in the sample, and to be derived from the light deriving unit 121b under a state in which the sample is treated as a core material and the chip 101 is treated as a clad material.

The detection unit 16 in the mobile terminal 14 is configured to detect the transmittance of the irradiated light through the detection and reaction unit 108 in the chip with a structure shown in FIG. 18 and FIG. 19.

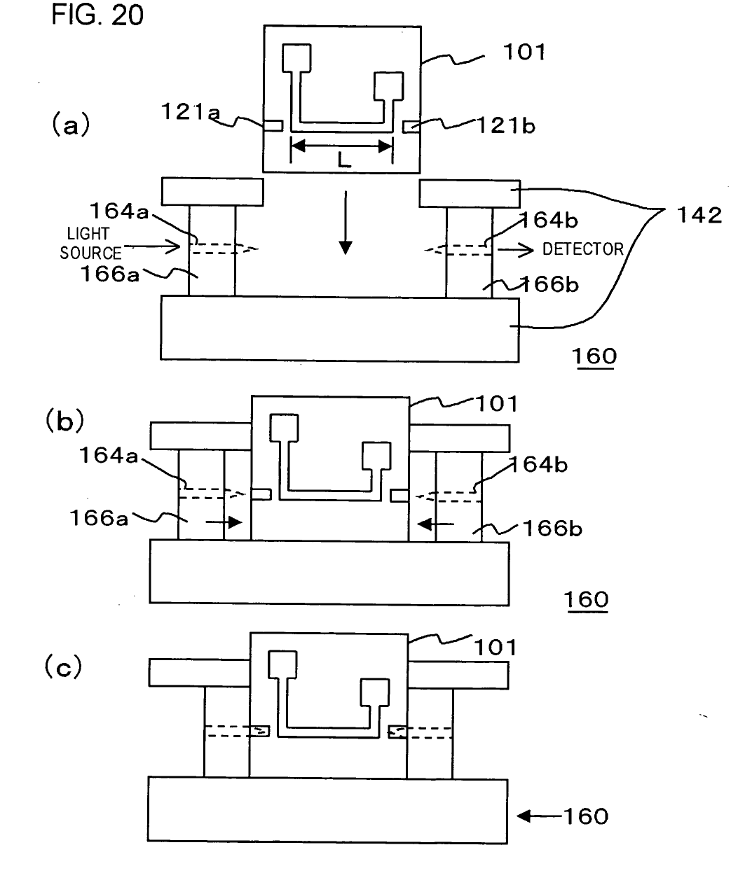

FIG. 20 is a view showing a connector which connects the chip 101 explained with referring to FIG. 19, and an outside light source and an outside detector. The mobile terminal 14 can be configured to include such connector.

The connector 160 includes a support body 142 which accommodates and supports the chip 101, a slide unit 166a and a slide unit 166b respectively holding an optical fiber 164a for light irradiation and an optical fiber 164b for light receiving.

As shown in FIG. 20(b), the slide unit 166a and the slide unit 166b hold the optical fiber 164a for light irradiation and the optical fiber 164b for light receiving in such a way that the optical fiber 164a for light irradiation and the optical fiber 164b for light receiving are respectively connected to the connection unit 121a and the connection unit 121b of the chip 101 when the chip 101 is accommodated in the support body 142 and the unit 166a and the unit 166b are respectively slided in the direction of arrows. Thereby, as shown in FIG. 20(c), the optical fiber 164a for light irradiation and the optical fiber 164b for light receiving can be configured to be respectively inserted into the connection unit 121a and the connection unit 121b of the chip 101. According to the above-described configuration, an optical path L can be increased along the detection and reaction unit 108 in the chip 101, and the element in the sample, which exists in the detection and reaction unit 108, can be accurately detected.

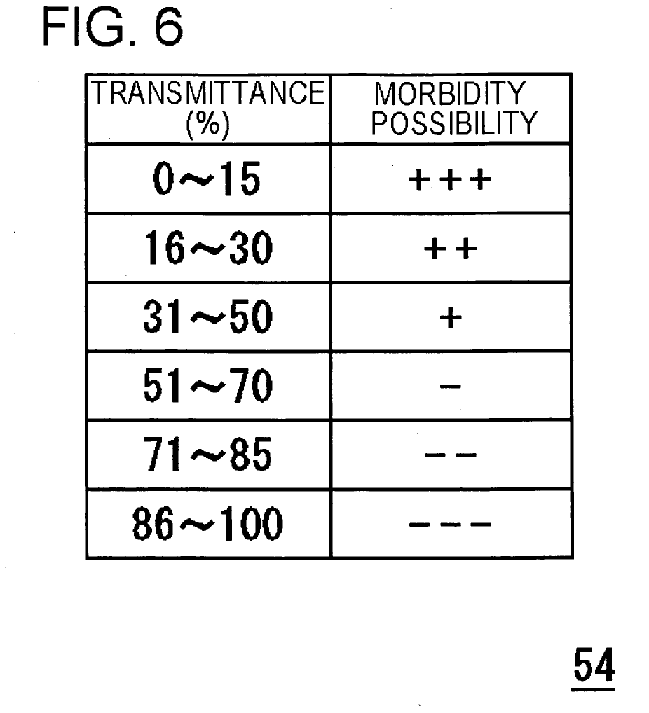

FIG. 6 is a view showing one example of a data structure for the analysis-information storage unit 54 shown in FIG. 2. Now, an example in which the analysis-information storage unit 54 stores comparison data as reference parameters will be explained. The reference parameters are set, based on data obtained by statistical processing of the measured transmittances and the degree of the morbidity with the body fluids of a test subject who has actually had close observation of a doctor are used as the sample, in a similar manner to the explanation referring to FIG. 3 to FIG. 5. Here, a morbidity possibility is set as “+++” when the transmittance is 0% to 15%, a morbidity possibility is set as “++” when the transmittance is 16% to 30%, a morbidity possibility is set as “+” when the transmittance is 31% to 50%, a morbidity possibility is set as “−” when the transmittance is 51% to 70%, a morbidity possibility is set as“−−” when the transmittance is 71% to 85%, and a morbidity possibility is set as “−−−” when the transmittance 86% to 100%. Here, the possibility of suffering from pollinosis, from highest to lowest, is shown with “+++”, “++”, “+”, “−”, “−−”, and “−−−”.

Here, the analysis-information updating unit 42 shown in FIG. 2 compares diagnosis results obtained by those of doctors and calculated results of the morbidity possibilities by the morbidity possibility calculation unit 36, updates the reference parameters in the analysis-information storage unit 54 when there is caused any gap between the calculated result and the diagnosis results. For example, the analysis-information updating unit 42 can update the set values in the analysis-information storage unit 54 in such a way that the morbidity possibility is made higher for each transmittance when there are generated many cases in which a user for whom the morbidity possibility calculation unit 36 determines that the morbidity possibility is low is diagnosed by a doctor as having pollinosis, in the case where the morbidity possibility calculation unit 36 calculates the morbidity possibility of a user, based on the comparison data as shown in FIG. 6.

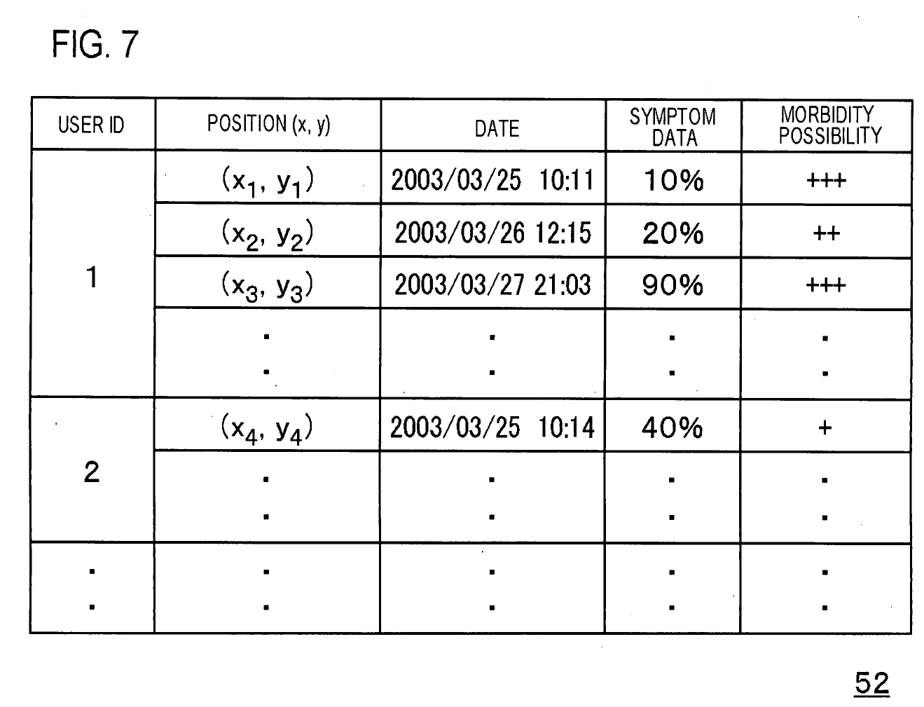

FIG. 7 is a view showing one example of a data structure for the data storage unit 52 shown in FIG. 2. The data storage unit 52 includes a user-ID column, a position (x, y) column, a date column, a symptom-data column, and a morbidity possibility column. The user-ID column holds user IDs each identifies the mobile terminal 14 of the user. The user ID may further include a general personal-identification information such as a servicing-agreement number and the number of a health-insurance ID card, and the analysis center 20 shall be required to legally acquire the above information. The position (x, y) column holds the positions for users, and the date column holds the dates on which the pieces of symptom data were made. The symptom-data column holds the symptom data obtained by the data-obtaining unit 26, and the morbidity possibility column holds the possibilities that each user is suffering from pollinosis calculated by the morbidity possibility calculation unit 36. Here, the position information is illustrated with the x-axis and the y-axis, but may be expressed by a place name or a building name.

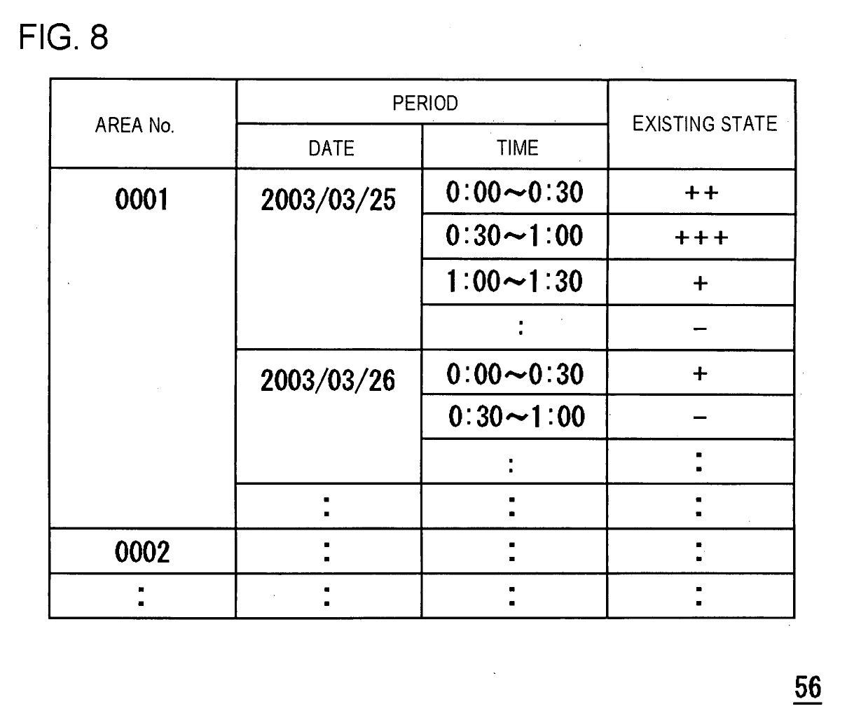

FIG. 8 is a view showing one example of a data structure for the estimation-result storage unit 56 shown in FIG. 2. The estimation-result storage unit 56 includes an area-No. column, a period column, and an existing state column. The area-No. column holds numbers by which predetermined areas are identified. The period column includes a date column and a time column. The existing state column holds existing states for a causative substance causing pollinosis in correspondence with the areas and periods. Here, the existing amount of the causative substance, from highest to lowest, is shown with, for example, “+++”, “++”, “+”, and “−”.

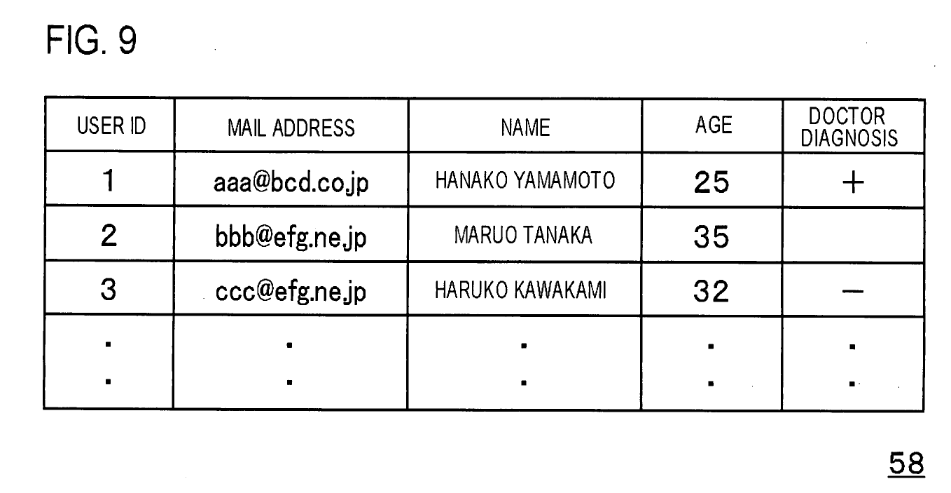

FIG. 9 is a view showing one example of a data structure for the user-information storage unit 58 shown in FIG. 2. The user-information storage unit 58 includes a user-ID column, a mail-address column, a name column, an age column, and a doctor-diagnosis column. The user-ID column holds user Ids, which identify each user. The mail-address column holds mail addresses of mobile terminals 14 for each user. The name column holds names of users, and the age column holds ages for each user. The doctor-diagnosis column holds information, for example, on whether a user has had close observation of a doctor in the past to know whether the user is suffering from pollinosis, and on whether the user has been diagnosed as having pollinosis when the user has had the close observation. Here, it is represented with, for example, “+” when a doctor diagnosed that the user was suffering from pollinosis, “−” when a doctor diagnosed that the user was not suffering from pollinosis, and a blank space when the user has not had a diagnosis by a doctor. For example, the user with a user ID of “1” is Hanako Yamamoto” 25 years old, her mail address is “aaa@bcd.co.jp”, and a doctor diagnosed that she was suffering from pollinosis. Here, the user information is required to be acquired in a legal manner.

Returning back to FIG. 2, the morbidity possibility calculation unit 36 can calculate the morbidity possibility with referring to a result diagnosed by a doctor, when calculating the morbidity possibility for a user. For example, the analysis-information storage unit 54 can further store comparison data for users who have been diagnosed as pollinosis by each doctor, in addition to the comparison data shown in FIG. 6. For users who have been diagnosed as pollinosis by each doctor, the morbidity possibilities can be set higher even when the transmittances are high. Although the comparison data is set in such a way that the morbidity possibility is calculated as “++” when the transmittance is 16% to 30% in FIG. 6, the comparison datamaybe set in such a way that the morbidity possibility is calculated as “+++” even when the transmittance is 16% to 30% for example for users who have been diagnosed as pollinosis by each doctor.

Moreover, the estimation processing unit 34 can estimate the existing states with considering the morbidity possibilities and results by doctor's diagnosis for each user, when estimating the existing state of the causative substance causing pollinosis. The existing amount of the causative substance can be estimated high in areas or periods, for example, in which a ratio of users diagnosed by doctors as pollinosis to users with a high morbidity possibility is high, and a ratio of users diagnosed by doctors as pollinosis to users with a low morbidity possibility is low.

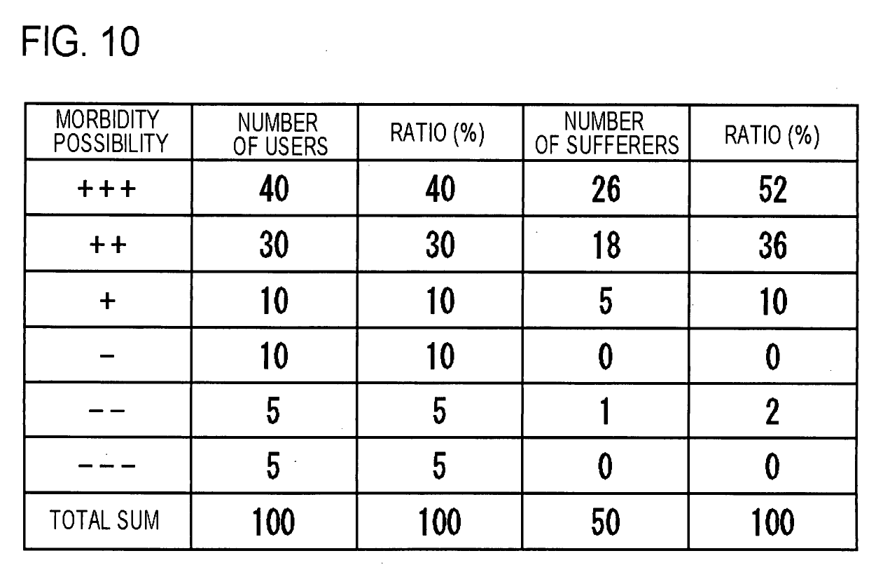

FIG. 10 is a view showing morbidity possibilities of a plurality of users in a predetermined area and a predetermined period in a statistical manner. The data obtaining unit 26 has obtained pieces of symptom data for a hundred users in the above area and period. Here, the number of users for whom the morbidity possibility has been calculated as “+++” is 40; the number of users calculated as “++” is 30; the number of users calculated as “+” is 10; the number of users calculated as “−” is 10; the number of users calculated as “−−” is 5; and the number of users calculated as “−−−” is 5. When the ratios of users with each morbidity possibility are calculated, using the total number of the users, the ratio for users with the calculated morbidity possibility of “+++” is 40%, the ratio for users calculated as “++” is 30%, the ratio for users calculated as “+” is 10%, the ratio for users calculated as “−” is 10%, the ratio for users calculated as “−−” is 5%, and the ratio for users calculated as “−−−” is 5%. The estimation processing unit 34 can estimate the existing state of the causative substance in the above area and period in correspondence with the ratio of users with the calculated morbidity possibility of, for example, “+++” or “++”. The analysis-information storage unit 54 stores information by which it is determined what kind of a standard is used for estimation of the existing state as well.

Moreover, the estimation processing unit 34 statistically processes the number of sufferers, among users, who have been diagnosed by doctors as pollinosis. Here, the total number of sufferers, among users, who have been diagnosed by doctors as pollinosis, is 50. Among the above total number of users, the number of users for which the morbidity possibility is calculated as “+++” in the morbidity possibility calculation unit 36 is 26; the number of users calculated as “++” is 18; the number of users calculated as “+” is 5; the number of users calculated as “−” is 0; the number of users calculated as “−−” is 1; and the number of users calculated as “−−−” is 0. When the ratios of users with each morbidity possibility are calculated, using the total number of the users, the ratio for users with the calculated morbidity possibility of “+++” is 52%, the ratio for users calculated as “++” is 36%, the ratio for users calculated as “+” is 10%, the ratio for users calculated as “−” is 0%, the ratio for users calculated as “−−” is 2%, and the ratio for users calculated as “−−−” is 0%. The estimation processing unit 34 can estimate the existing state of the causative substance in the above area and period, with considering the ratio of users for whom the morbidity possibility has been calculated as “+++” or “++” by the morbidity possibility calculation unit 36, to users who have been diagnosed by doctors as a sufferer of pollinosis.

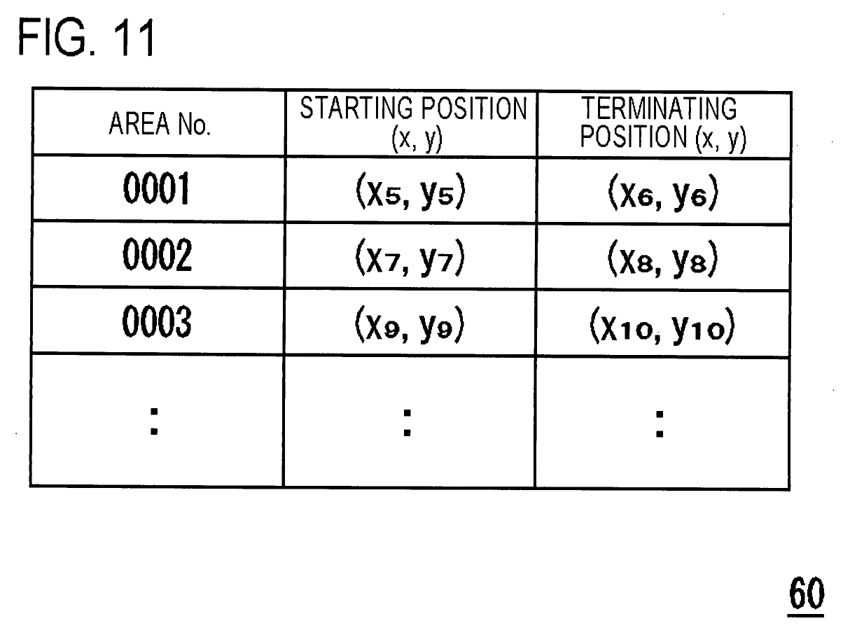

FIG. 11 is a view showing one example of a data structure for the area-information storage unit 60 shown in FIG. 2. The area-information storage unit 60 includes an area-No. column, a starting-position (x, y) column, and a terminating-position (x, y) column. The area-No. column corresponds to the area-No. column shown in FIG. 6, and each area is set as a range enclosed by x-axes respectively passing the starting position and the terminating position and y-axes respectively passing the starting position and the terminating position.

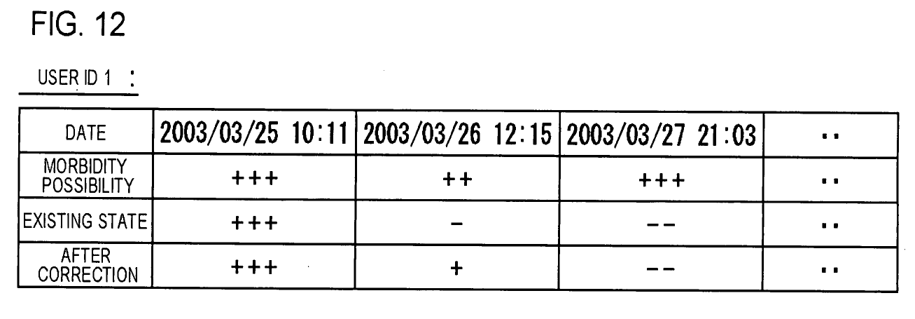

FIG. 12 is a view showing relations between the morbidity possibilities calculated by the morbidity possibility calculation unit 36 based on the symptom data for a certain user and the existing states of the causative substance causing pollinosis on the corresponding dates and at the corresponding positions. The morbidity possibility is calculated as “+++” based on the symptom data which was acquired from the user with a user ID of “1”, for example, at 10:11 am on Mar. 25, 2003. At this time, the existing state of the causative substance causing pollinosis at a position at which this user stands is estimated as “+++”. Accordingly, the correction processing unit 38 leaves the morbidity possibility at this time as “+++”. On the other hand, the morbidity possibility is calculated as “++”, based on the symptom data, which was acquired from this user at 12:15 on Mar. 26, 2003. At this time, the existing state of the causative substance causing pollinosis at a position at which this user stands is estimated as “−”. Accordingly, the correction processing unit 38 corrects the morbidity possibility at this time, for example, as “+”. Thus, the correction processing unit 38 corrects the morbidity possibility of this user, based on the existing states of the causative substance for the areas and the periods in correspondence with the morbidity possibilities of the same user on a plurality of dates or at a plurality of positions.

For example, when the calculated morbidity possibility based on the symptom data is high, and the existing amount of the causative substance is also high, the possibility of suffering from pollinosis may be calculated high. On the other hand, when the calculated morbidity possibility based on the symptom data is high although the existing amount of the causative substance causing pollinosis is low, it can be calculated that a possibility of suffering not from pollinosis, but from a disease such as a cold, which is not related to the existence of the causative substance is high. Moreover, when the existing amount of the causative substance causing pollinosis is low, the possibility that the symptom of pollinosis is not shown is high even for the user actually suffering from pollinosis, therefore, it can be calculated that a possibility of suffering from pollinosis is high even the calculated morbidity possibility based on the symptom data is low. Moreover, it can be calculated that a possibility of not suffering from pollinosis is high when the calculated morbidity possibility based on the symptom data is low even the existing amount of the causative substance causing pollinosis is high.

Moreover, the correction processing unit 38 may correct the morbidity possibility based on the correspondences between high/low relation of the morbidity possibilities calculated based on the symptom data with high/low relation of the existing amounts of the causative substance causing pollinosis, not individually comparing the morbidity possibilities with the existing states on each date.

When the symptom data is sent to the analysis center 20, a user is not always required to detect the existence of a feature component with the chip 101 after collecting the body fluids of the user. After the body fluids are collected once, and the existence of the feature component is detected with the chip 101, information on whether a similar symptom to the symptom at collecting the body fluids is generated or not, or information on whether the symptom is milder or severer than the symptom that was caused at collecting the body fluids may be transmitted to the analysis center 20 as the symptom data in a form of answers to a questionnaire sheet. When the information is received, the morbidity possibility calculation unit 36 can calculate the morbidity possibility of pollinosis in the analysis center 20, based on the answers of the user to the questionnaire sheet, and the symptom data which has been obtained when the body fluids were collected.

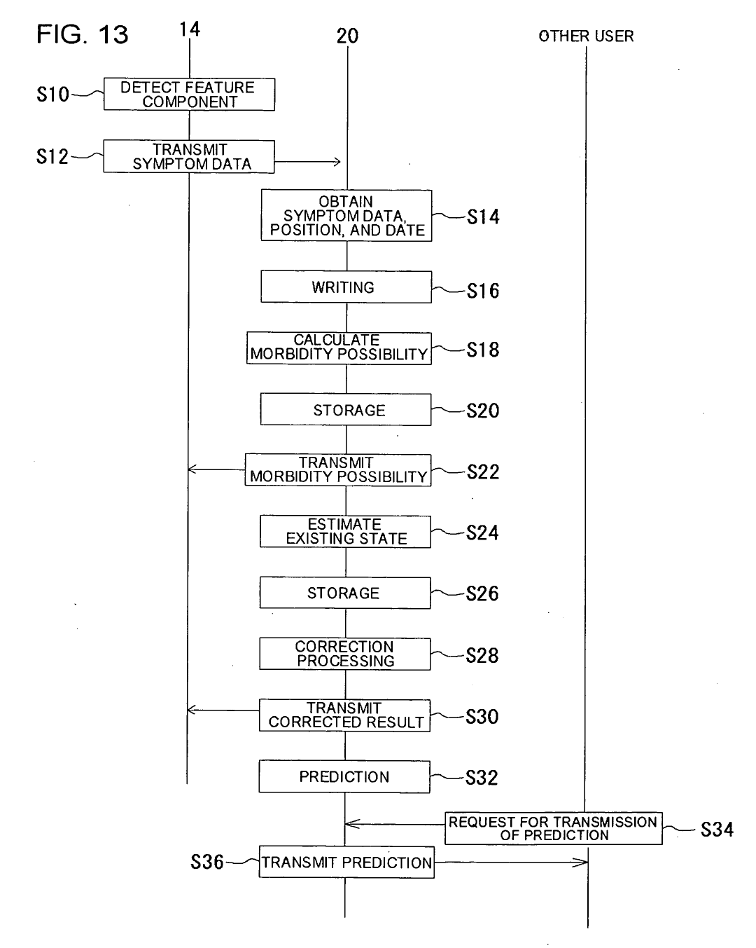

FIG. 13 is a flowchart showing processing procedures in the mobile terminal 14 and the analysis center 20 according to this embodiment.

In the first place, a user uses the chip 101 to form the color of a feature component in the mobile terminal 14, and the detection unit 16 in the mobile terminal 14 detects the feature component (S10). The mobile terminal 14 transmits the detected result of the feature component to the analysis center 20 as the symptom data representing the symptom of the user (S12). In the analysis center 20, the data obtaining unit 26 obtains the symptom data, the date on which the symptom data was made, and the position at which the symptom data was made (S14). The data obtaining unit 26 writes the symptom data into the data storage unit 52 in correspondence with the position and the date (S16). Based on the symptom data, the morbidity possibility calculation unit 36 calculates the possibility that the user is suffering from pollinosis, referring to the analysis-information storage unit 54 (S18), and stores the morbidity possibility in the data storage unit 52 (S20).

The estimation processing unit 34 estimates the existing states of the causative substance causing pollinosis for each area and for each of predetermined periods, based on the data transmitted form a plurality of users (S24), and stores the results in the estimation-result storage unit 56 (S26). Referring to the data storage unit 52 and the estimation-result storage unit 56, the correction processing unit 38 corrects the morbidity possibility of each user, based on the morbidity possibility and the existing state of the causative substance, which have been calculated in the morbidity possibility calculation unit 36 (S28). The transmission processing unit 40 transmits the morbidity possibility corrected by the correction processing unit 38 to the mobile terminal 14 (S30). At this time, the transmission processing unit 40 may also transmit the morbidity possibility before correction to the mobile terminal 14. Moreover, the estimation processing unit 34 predicts the existing state of the causative substance (S32). Here, a method by which the existing state is predicted may include, for example, a method by an auto regressive model (Refer to, for example, “Practice of Time Series Analysis I”, supervised by Hirotsugu Akaike, published by Asakura Publishing Company (ASAKURA SHOTEN), Tokyo, 1994; and “Practice of Time Series Analysis II”, supervised by Hirotsugu Akaike, published by Asakura Publishing Company (ASAKURA SHOTEN), Tokyo, 1995). When another user makes a request to transmit the existing state of the causative substance and the prediction of the state (S34), the delivery processing unit 44 delivers the existing state of the causative substance and the prediction of the state to the user (S36). The delivery processing unit 44 may obtain a user ID of the user, together with the request to transmit the existing state of the causative substance and the prediction of the state, from the user, and may transmit information in accordance with the history of the user, such as the existing state of an antigen causing an allergic disease to the user, by referring to the user-information storage unit 58. Moreover, the delivery processing unit 44 may disclose the existing state of the causative substance, and the prediction of the state on a web page and the like. Here, correction by the correction processing unit 38 may also be performed after the morbidity possibility is transmitted to the mobile terminal 14 as shown at the step 22.

As the analysis center 20 according to this embodiment quickly calculates whether a user is suffering from pollinosis, based on the symptom data which the user has transmitted from the mobile terminal 14, and transmits the estimated result to the mobile terminal 14, the user can quickly know the possibility that the user is suffering from pollinosis. Moreover, the existing state of the causative substance causing pollinosis is estimated, based on piece of symptom data, which have been received from a number of users, and the estimated existing state can be feedbacked to calculation of the morbidity possibility for a user, or can be provided to other users. Thereby, a number of users can accurately know the existing state of the causative substance causing pollinosis. Thus, the morbidity possibility for pollinosis can be accurately calculated, because the possibility that a user is suffering from pollinosis is corrected according to not only the symptom of each user, but also to the existing state of the causative substance and the like, as described above.

Not only a user who buys the chip 101 and transmits the symptom data, but also other people can know the existing state of the causative substance causing pollinosis, therefore the person can judge whether he or she is suffering from pollinosis, by comparing his or her symptom and the existing state of the causative substance. The information on existing state of such a causative substance may be provided on a chargeable basis, and by using money paid by such the users for manufacturing the chip 101, the chip 101 can be manufactured at a low cost. Moreover, the information on existing state of the causative substance can be provided together with advertisement information when the state is provided through a web page and the like. Thereby, the advertising expenses can be used for manufacturing the chip 101.

### Second Embodiment

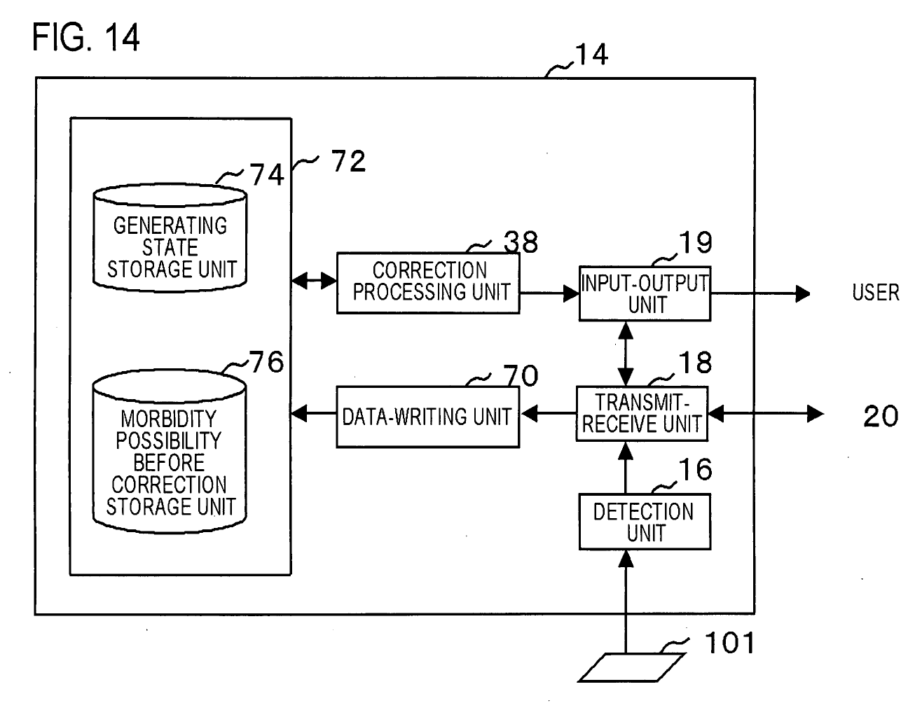

FIG. 14 is a block diagram showing a configuration for a mobile terminal 14 according to a second embodiment of the present invention.

This embodiment is different from the first embodiment in a point that the mobile terminal 14 has the correction processing unit 38. In this embodiment as well, the analysis center 20 has a similar configuration to that of the first embodiment shown in FIG. 2. Here, the analysis center 20 is not required to include the correction processing unit 38.

The mobile terminal 14 further includes the correction processing unit 38, a data writing unit 70, and a storage unit 72, in addition to those in the configuration which is explained in the first embodiment, referring to FIGS. 2, 4, and 5. The storage unit 72 includes a generating-state storage unit 74, and a unit for storing morbidity possibility before correction 76. In this embodiment, component elements similar to those of the first embodiment are denoted by the same reference numbers as those in the first embodiment, and detailed description is omitted sometimes.

In this embodiment, the transmit-receive unit 18 receives a morbidity possibility calculated in the morbidity possibility calculation unit 36, and an existing state estimated in the estimation processing unit 34 from the analysis center 20. The data writing unit 70 writes the morbidity possibility and the existing state received by the transmit-receive unit 18 into the unit for storing morbidity possibility before correction 76 and the generating-state storage unit 74. The correction processing unit 38 reads out the morbidity possibilities, and the existing states of the causative substance at corresponding positions and dates from the unit for storing morbidity possibility before correction 76 and the generating-state storage unit 74, and corrects the morbidity possibility that a user is suffering from pollinosis with considering them.

Thereby the user of the mobile terminal 14 can know his or her morbidity possibility for pollinosis, based on the morbidity possibility transmitted from the analysis center 20, and, at the same time, can know the morbidity possibility corrected by the correction processing unit 38 according to the existing states of the causative substance to detect the morbidity possibility in a more accurate manner. After the body fluids are collected once, and the existence of the feature component is detected with the chip 101, and the morbidity possibility is received from the analysis center 20, the morbidity possibility for pollinosis can be determined in the time series at the side of the mobile terminal 14, considering information on whether a similar symptom to the symptom at collecting the body fluids is generated or not, and the existing state of the causative substance transmitted from the analysis center 20.

The invention has been explained, based on the embodiment as described above. It will be appreciated by persons skilled in the art that the embodiment is to be considered as illustrative, various kinds of variations may be possible, and the above variations may be within the scope and equivalence of the appended claims.

For example, the mobile terminal 14 may have a configuration in which the detection unit 16 is not included. In this case, for example, a configuration in which the user of the mobile terminal 14 receives a questionnaire sheet from the analysis center 20, and transmits answers to the questionnaire sheet to the analysis center 20 as symptom data may be possible. The correction processing unit 38 provided in the analysis center 20 or in the mobile terminal 14 can correct the morbidity possibility, based on the morbidity possibilities which has been determined, based on a plurality of pieces of symptom data for different positions or different periods, and the existing states of the causative substance for pollinosis at positions and periods in correspondence with the above morbidity possibilities. Although it is difficult to distinguish pollinosis from other diseases such as a cold only by the answers to the questionnaire sheet, by correcting the morbidity possibility, based on the relations between the existing states of the causative substance causing pollinosis and the morbidity possibilities, it is possible to accurately determine whether a user is suffering from pollinosis.

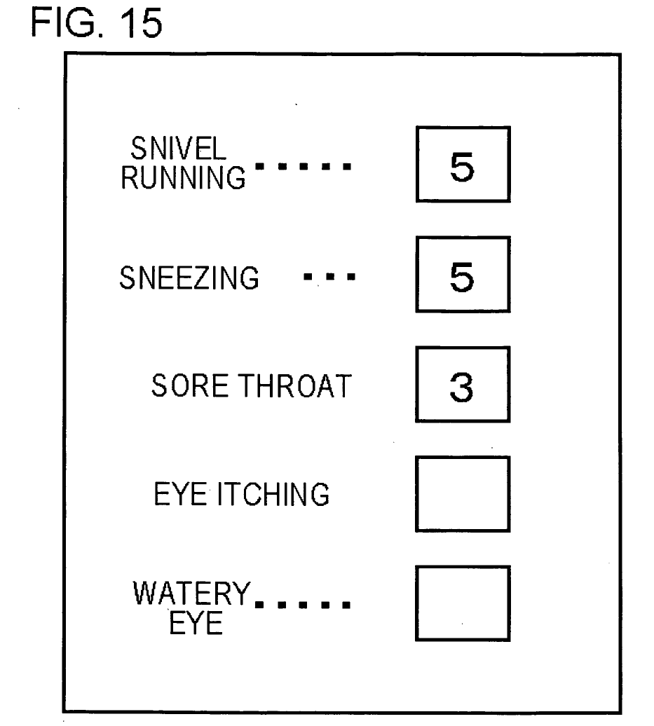

FIG. 15 is a view showing one example of a questionnaire sheet. Here, answers to the questionnaire sheet including the following questions are input, based on, for example, a five-level rating system; whether “snivel-running” is caused or not; whether “sneezing” is generated or not; whether “sore throat” is caused or not; whether there is “itching” in the eye or not; and whether is “watery eye” is caused or not.

Furthermore, a color chart defining correspondences between the quantities of forming a color caused by a coloring agent in the chip 101 and the existing amount of the causative substance can be distributed, together with the chip 101. In this case, there may be possible a configuration in which a user is required to determine the existing amount of the causative substance, based on the color chart, and to input the determined result into the mobile terminal 14 for transmission of the result to the analysis center 20.

Moreover, there may be possible another form in which a user transmits the symptom data from a fixed terminal such as a personal computer although explanation has been made in the above-described embodiment assuming that a user transmits symptom data from the mobile terminal 14. In this case, a user inputs a position at which and a date on which a symptom in the symptom data was caused, using the terminal, and transmits the input position and the date to the analysis center 20 in correspondence with the symptom data. Thereby, the analysis center 20 can estimate the existing state of the causative substance causing pollinosis, and can correct the morbidity possibility according to the existing state of the causative substance in a similar manner to that of the above-described embodiment.

Furthermore, the analysis center 20 may include means for obtaining the scattering states of each of a plurality of causative substances, and the estimation processing unit 34 may estimate the existing states of each causative substance, considering the above scattering states, and information on sufferers for whom the causative substance is specified by diagnosis of a doctor. In this case, the analysis center 20 may estimate causative substances by which each user develops a symptom of a disease, considering the existing states of each causative substance and the morbidity possibilities of each user. Thereby, it is possible to estimate an antigen causing an allergic disease by a simple and cheap method.

Here, explanation has been made in the above-described embodiment, using pollinosis as an example, but morbidity examination for a severe acute respiratory syndrome (SARS), influenza examination, house dust examination and the like can be realized by introducing a primary antibody, which is appropriately changed for use in a measuring method such as an EIA, an ELISA, or an immune chromatography, into the chip 101.

Moreover, identification information may be given to the chip 101, and the identification information for the chip 101 may be simultaneously transmitted from the mobile terminal 14 to the analysis center 20, together with the symptom data and the position data. A service by which information on the morbidity possibility, and that on the prediction of the possibility are provided only to users who has bought the chip 101 from a specific chip supplier can be realized, and, in addition, product control of the chip 101 can be executed by including information on the supplier of the chip 101, the kind of the chip 101, and a manufacturer's serial number as the identification information.

1. A diagnostic support system which comprises a mobile terminal and an analysis center which are connected to each other through a network, and judges a morbidity possibility that a user holding said mobile terminal is suffering from a disease, wherein
said mobile terminal comprises:
a detection unit which detects whether a feature component representing the feature of the morbidity of said disease is included in a sample collected from said user or not; and
a transmission processing unit which transmits the detected result by said detection unit to said analysis center as symptom data representing the symptom of said user, and
said analysis center comprises:
a data obtaining unit which obtains said symptom data in correspondence with the position of said mobile terminal at which said symptom data was transmitted from said mobile terminal;
a morbidity possibility calculation unit which calculates the morbidity possibility that said user is suffering from said disease, based on said symptom data and a reference parameter representing a feature caused in a sufferer of said disease; and
an estimation processing unit which estimates the existing state of sufferers of said disease for each area, based on said morbidity possibility of a plurality of said users and corresponding said position.

2. The diagnostic support system according to claim 1, wherein
said analysis center further comprises a map-information storage unit which stores map information including information on buildings, and
said estimation processing unit estimates the existing state of sufferers of said disease for each area defined by each building, based on said morbidity possibility of said plurality of users, corresponding said position, and information on a building included in said map information.

3. The diagnostic support system according to claim 1, wherein
said estimation processing unit estimates the existing state of the causative substance causing said disease for each area, based on the existing state of sufferers of said disease.

4. The diagnostic support system according to claim 1, wherein
said data obtaining unit obtains said symptom data also in correspondence with a date on which said symptom data was made, and
an estimation processing unit estimates said existing state for each area and each period, based on said morbidity possibility for said plurality of users, and corresponding said position and said date.

5. The diagnostic support system according to claim 4, wherein
said analysis center further comprises:
a correction processing unit which corrects said morbidity possibility according to said existing state in an area including corresponding said position and in a period including said date; and
a delivery processing unit which delivers said morbidity possibility corrected by said correction processing unit to said mobile terminal.

6. The diagnostic support system according to claim 4, wherein
said analysis center further comprises a delivery processing unit which delivers said morbidity possibility calculated based on said symptom data, together with said existing state in an area including corresponding said position and in a period including said date, to said mobile terminal.

7. The diagnostic support system according to claim 6, wherein
said mobile terminal further comprises:
a receiving unit which receives said morbidity possibility and said existing state in an area including corresponding said position; and
a correction processing unit which corrects said morbidity possibility according to said existing state.

8. A diagnostic support system which judges the morbidity possibility of a disease, comprising:
a data obtaining unit which obtains symptom data representing the symptom of a test subject in correspondence with a position at which and date on which said symptom data was made;
a morbidity possibility calculation unit which calculates a morbidity possibility that said test subject is suffering from said disease, based on said symptom data and reference parameters representing the features generated in sufferers of said disease;
an estimation processing unit which estimates the existing state of sufferers of said disease for each area and each period; and
a correction processing unit which corrects said morbidity possibility according to said existing state at said position and on said date.

9. The diagnostic support system according to claim 8, further comprising a map-information storage unit which stores map information including information on buildings, wherein
said estimation processing unit estimates the existing state of sufferers of said disease for each area defined by each building, based on said morbidity possibilities of said plurality of users, corresponding said positions, and information on buildings included in said map information.

10. The diagnostic support system according to claim 9, further comprising:
a display processing unit which displays the existing state estimated by said estimation processing unit, together with said map information; and
a selection accepting unit which accepts selection of a point included in map information displayed by said display processing unit from a user, wherein
said display processing unit displays the existing state of sufferers at a point selected by said user in correspondence with a date.

11. The diagnostic support system according to claim 9, wherein
said map information includes information on each room in buildings,
said estimation processing unit estimates the existing states of sufferers of said disease for each area defined by each room, based on said morbidity possibilities of said plurality of users, corresponding said positions, and information on each room in buildings included in said map information,
said diagnostic support system further comprises:
a display processing unit which displays the existing state estimated by said estimation processing unit, together with buildings included in said map information; and
a selection accepting unit which accepts selection of a point defined by each of said rooms included in map information from a user, and
said display processing unit displays the existing state of sufferers in the room selected by said user.

12. The diagnostic support system according to claim 8, wherein
said estimation processing unit estimates the existing state of the causative substance causing said disease for each area and each period, based on the existing state of sufferers of said disease.

13. The diagnostic support system according to claim 8, wherein
said data obtaining unit obtains pieces of said symptom data in the same area and in the same period from a plurality of test subjects,
said morbidity possibility calculation unit calculates said morbidity possibilities for each of said pieces of symptom data of said plurality of test subjects, and
said existing state obtaining unit estimates said existing states, for said area and said period, based on said morbidity possibilities of said plurality of test subjects.

14. The diagnostic support system according to claim 8, wherein
said data obtaining unit obtains a plurality of pieces of said symptom data in different areas, or different periods from said test subjects,
said morbidity possibility calculation unit calculates said morbidity possibilities for each of said plurality of pieces of symptom data, and
said correction processing unit corrects said morbidity possibilities, based on relations between said plurality of morbidity possibilities and said plurality of existing states for respectively corresponding said areas and said periods.

15. The diagnostic support system according to claim 8, wherein
said data obtaining unit obtains data indicating whether a feature component representing the feature of the morbidity of a disease exists in a sample collected from said test subject or not, and
said morbidity possibility calculation unit calculates the morbidity possibility that said test subject is suffering from said disease, based on the data indicating whether said feature component exists or not, and on said reference parameters.

16. The diagnostic support system according to claim 15, wherein
said data obtaining unit obtains information on whether said test subject develops a similar symptom to that developed at the time when said test subject collected said sample or not, in an area different from the area in which the data indicating whether said feature component exists or not was acquired or a period different from the period in which the data indicating whether said feature component exists or not was acquired,
said morbidity possibility calculation unit calculates said morbidity possibility of said test subject for the time at which said information was acquired from said test subject, based on the data indicating whether said feature component exists or not, and on said information, and
said correction processing unit corrects said morbidity possibility, based on relations between said plurality of morbidity possibilities and said plurality of existing states for the causative substance in said areas and said periods, which are corresponding to said possibilities.

17. The diagnostic support system according to claim 16, wherein
said test subject is a user who uses a mobile terminal,
said data obtaining unit obtains said symptom data from said mobile terminal, and a position of said mobile terminal for the time, at which said symptom data was transmitted from said mobile terminal, in correspondence with said symptom data.

18. A diagnostic support system calculating the morbidity possibility of a disease, comprising:
a data obtaining unit which obtains symptom data representing the symptom of a test subject in correspondence with the position at which and with the date on which said symptom data was made;
a morbidity possibility calculation unit which calculates a morbidity possibility that said test subject is suffering from said disease, based on said symptom data and reference parameters representing a feature which is generated in sufferers of said disease; and
an estimation processing unit which predicts the existing state of sufferers of said disease for each area or each period, based on said morbidity possibilities calculated said plurality of pieces of symptom data in different areas or different periods.

19. A mobile terminal which is used for a diagnostic support system provided with an analysis center which judges a morbidity possibility that a user is suffering from a disease, based on symptom data representing the symptom of the user, comprising:
a detection unit which detects whether a feature component representing the feature of the morbidity of said disease exists in a sample collected from said user or not; and
a transmission processing unit which transmits the detected result by said detection unit to said analysis center as symptom data representing the symptom of said user.

20. The mobile terminal according to claim 19, further comprising;
a receiving unit which receives said morbidity possibility judged based on said symptom data and the existing state of a causative substance causing said disease in a position at which and on a data on which said sample was collected; and
a correction processing unit which corrects said morbidity possibility according to said existing state.

21. The mobile terminal according to claim 20, further comprising:
an input accepting unit which accepts, from a user, input of information on whether said user develops a similar symptom to that developed at the time when said user collected said sample or not, in an area different from the area where said user collected said sample or a period different from the period in which said user collected said sample, and
a morbidity possibility calculation unit which calculates said morbidity possibility of said user for a time at which said information was obtained from said user, based on said morbidity possibility determined based on said symptom data, and on said information, wherein
said receiving unit also receives said existing state in a position at which and on a date on which said user input said information, and
said correction processing unit corrects said morbidity possibilities, based on relations between said plurality of morbidity possibilities and said plurality of existing states at said positions and on said dates respectively corresponding to said possibilities.
